(19) United States
(12) Patent Application Publication
(10) Pub. No.: US 2023/0154618 A1
(43) Pub. Date: May 18, 2023

(54) Bayesian Approach For Tumor Forecasting

(71) Applicant: H. LEE MOFFITT CANCER CENTER AND RESEARCH INSTITUTE, INC., Tampa, FL (US)

(72) Inventors: Heiko Enderling, New Tampa, FL (US); Stefano Pasetto, Tampa, FL (US)

(21) Appl. No.: 18/055,956

(22) Filed: Nov. 16, 2022

Related U.S. Application Data
(60) Provisional application No. 63/279,994, filed on Nov. 16, 2021.

(51) Int. Cl. G16H 50/20; G16H 10/60

(52) U.S. Cl. CPC G16H 50/20; G16H 10/60

(57) ABSTRACT

Systems and methods utilizing a Bayesian framework for tumor forecasting are described herein. An example method may include: inputting a plurality of patient data for a patient into a multi-model framework; predicting, using the multi-model framework, a probability of a given treatment producing a given outcome for the patient; and outputting an assessment for the given treatment.

### STATEMENT REGARDING FEDERALLY FUNDED RESEARCH

This invention was made with government support under Grant no. 1R21CA234787-01A1 and U54CA143970-05 awarded by the National Institutes of Health/National Cancer Institute. The government has certain rights in the invention.

### CROSS-REFERENCE TO RELATED APPLICATIONS

This application claims priority to and incorporates by reference herein U.S. Pat. Application Serial No. 63/279,994 entitled BAYESIAN APPROACH FOR TUMOR FORECASTING, the contents of which is hereby incorporated by this reference in its entirety as if fully set forth herein.

### BACKGROUND

Tumor boards define the optimal clinical pathway for a patient daily. To achieve this goal, they require the capability to account for the patient’s current state, their anamnesis, the medical literature, and the hospital/clinic facility’s constraints in the rapid and dynamical context of a meeting. The idea to support this complex decisional process is advanced with a logic-founded statistical tool tailored to the tumor board necessities. Implementation of a cloud-computing service based on the Bayesian model comparison framework is proposed to support the tumor board decisional process. This tool will indicate the literature-known most successful clinical path to the closest clinical patient case under exam.

Embodiments of the present disclosure explore how to decide on the optimal treatment for a patient. The approach described herein aims to rate tumor-board-preselected optimal treatments with a Bayesian statistical tool rather than determine optimal treatment through externally specific indexes.

The importance of the Bayesian model comparison as a useful statistical framework in the tumor board decisional process is highlighted herein. Its ability to naturally weigh a patient’s state and medical doctors’ knowledge represents critical complementary support to oncology work.

The tool is designed to support a tumor board decisional process. Throughout the access to the proposed cloud-computing system, an oncologist will be able to insert the patient’s information and receive the most successful therapeutic path that has already been applied in the literature.

Ideally, specific treatment for a cancer patient is decided by a multidisciplinary tumor board, integrating prior clinical experience, published data, and patient-specific factors to develop a consensus on an optimal therapeutic strategy. However, many oncologists lack access to a tumor board, and many patients have incomplete data descriptions, so that tumor boards must act on imprecise criteria. These limitations may be addressed through a flexible but rigorous mathematical tool that can define the probability of success of given therapies and be made readily available to the oncology community. Here, a Bayesian approach to tumor forecasting using a multi-model framework is presented that can predict patient-specific response to different targeted therapies even when historical data are incomplete. The Bayesian decision theory’s integrative power permits the simultaneous assessment of a range of therapeutic options. This methodology, built upon a robust and well-established mathematical framework, can play a crucial role in supporting patient-specific clinical decisions by individual oncologists and multi-specialty tumor boards.

### SUMMARY

An example method may include: inputting a plurality of patient data for a patient into a multi-model framework; predicting, using the multi-model framework, a probability of a given treatment producing a given outcome for the patient; and outputting an assessment for the given treatment.

In some implementations, an example apparatus comprising at least one processor, at least one memory including computer program code for at least one program, and a network interface is provided. In some implementations, the at least one memory and the computer program code configured to, with the at least one processor, cause the apparatus to: input a plurality of patient data for a patient into a multi-model framework; predict, using the multi-model framework, a probability of a given treatment producing a given outcome for the patient; and output an assessment for the given treatment.

In some implementations, a computer program product comprising at least one non-transitory computer-readable storage medium having computer-executable program code portions stored therein is provided. In some implementations, the computer-executable program code portions comprise program code instructions, the computer program code instructions, when executed by a processor of a computing entity, are configured to cause the computing entity to at least: input a plurality of patient data for a patient into a multi-model framework; predict, using the multi-model framework, a probability of a given treatment producing a given outcome for the patient; and output an assessment for the given treatment.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is an example computing device.

FIGS. 2A-B are schematic diagrams according to implementations described herein

FIGS. 3A-D are schematic diagrams according to implementations described herein.

FIG. 4 is a schematic diagram according to implementations described herein.

FIG. 5 is a schematic diagram according to implementations described herein.

FIGS. 6A-B are schematic diagrams according to implementations described herein.

FIG. 7 is a schematic diagram according to implementations described herein.

FIGS. 8A-B are schematic diagrams according to implementations described herein.

FIGS. 9A-C are schematic diagrams according to implementations described herein.

FIGS. 10A-B are schematic diagrams according to implementations described herein.

FIGS. 11A-D are schematic diagrams according to implementations described herein.

FIGS. 12A-D are schematic diagrams according to implementations described herein.

FIGS. 13A-D are schematic diagrams according to implementations described herein.

FIGS. 14A-D are schematic diagrams according to implementations described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

Embodiments of the present disclosure present an example method. The example method may include: inputting a plurality of patient data for a patient into a multi-model framework; predicting, using the multi-model framework, a probability of a given treatment producing a given outcome for the patient; and outputting an assessment for the given treatment.

In some implementations, the multi-model framework comprises a Bayesian statistical model.

In some implementations, the Bayesian statistical model is configured to analyze respective predictions of a plurality of models of the multi-model framework.

In some implementations, wherein the patient data comprises at least one of demographic data, clinical data, laboratory data, histological feature data, comorbidity data, and medication data.

In some implementations, the given treatment comprises surgery, radiotherapy, chemotherapy, immunotherapy, or combinations thereof.

In some implementations, the given outcome comprises at least one of tumor burden, tumor local control, progression-free survival for a period of time, and relapse-free survival for a period of time.

In some implementations, the multi-model framework is implemented as a cloud-computing service or system.

In some implementations, the method further comprises recommending the given treatment for the patient.

In some implementations, the method further comprises administering the given treatment to the patient.

In some implementations, an example apparatus comprising at least one processor, at least one memory including computer program code for at least one program, and a network interface is provided. In some implementations, the at least one memory and the computer program code configured to, with the at least one processor, cause the apparatus to: input a plurality of patient data for a patient into a multi-model framework; predict, using the multi-model framework, a probability of a given treatment producing a given outcome for the patient; and output an assessment for the given treatment.

In some implementations, the multi-model framework comprises a Bayesian statistical model.

In some implementations, the Bayesian statistical model is configured to analyze respective predictions of a plurality of models of the multi-model framework.

In some implementations, the patient data comprises at least one of demographic data, clinical data, laboratory data, histological feature data, comorbidity data, and medication data.

In some implementations, the given treatment comprises surgery, radiotherapy, chemotherapy, immunotherapy, or combinations thereof.

In some implementations, the given outcome comprises at least one of tumor burden, tumor local control, progression-free survival for a period of time, and relapse-free survival for a period of time.

In some implementations, the multi-model framework is implemented as a cloud-computing service or system.

In some implementations, the at least one memory and the computer program code configured to, with the at least one processor, cause the apparatus to: recommend the given treatment for the patient.

In some implementations, the at least one memory and the computer program code configured to, with the at least one processor, cause the apparatus to: administer the given treatment to the patient.

In some implementations, a computer program product comprising at least one non-transitory computer-readable storage medium having computer-executable program code portions stored therein is provided. In some implementations, the computer-executable program code portions comprise program code instructions, the computer program code instructions, when executed by a processor of a computing entity, are configured to cause the computing entity to at least: input a plurality of patient data for a patient into a multi-model framework; predict, using the multi-model framework, a probability of a given treatment producing a given outcome for the patient; and output an assessment for the given treatment.

In some implementations, the multi-model framework comprises a Bayesian statistical model.

In some implementations, the Bayesian statistical model is configured to analyze respective predictions of a plurality of models of the multi-model framework.

In some implementations, the patient data comprises at least one of demographic data, clinical data, laboratory data, histological feature data, comorbidity data, and medication data.

In some implementations, the given treatment comprises surgery, radiotherapy, chemotherapy, immunotherapy, or combinations thereof.

In some implementations, the given outcome comprises at least one of tumor burden, tumor local control, progression-free survival for a period of time, and relapse-free survival for a period of time.

In some implementations, the multi-model framework is implemented as a cloud-computing service or system.

In some implementations, the computer program code instructions, when executed by a processor of a computing entity, are configured to cause the computing entity to at least: recommend the given treatment for the patient.

In some implementations, the computer program code instructions, when executed by a processor of a computing entity, are configured to cause the computing entity to at least: administer the given treatment to the patient.

### Example Computing Device

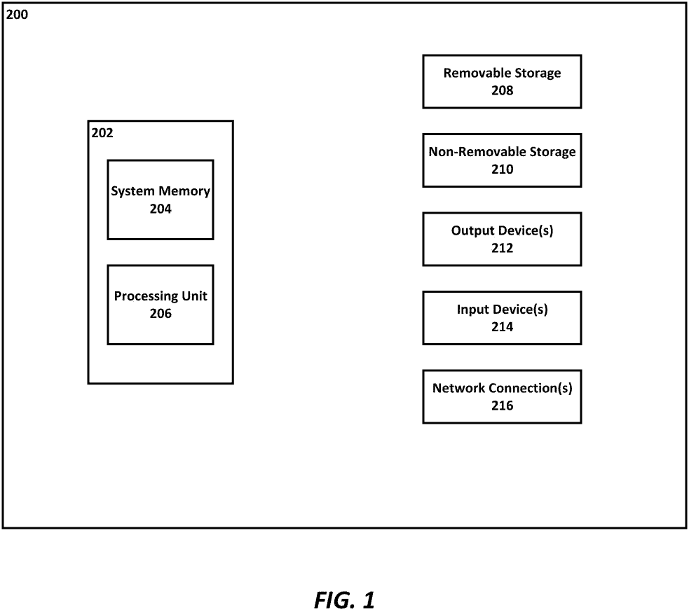

It should be appreciated that the logical operations described herein with respect to the various figures may be implemented (1) as a sequence of computer implemented acts or program modules (i.e., software) running on a computing device (e.g., the computing device described in FIG. 1), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 1, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random-access memory (RAM)), non-volatile (such as read-only memory (ROM), flash memory, etc.), or some combination of the two. This most basic configuration is illustrated in FIG. 1 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

The treatment or treatment combinations for individual cancer patients are often determined by a tumor board of physicians from different specialties such as surgery, pathology, medical oncology, and radiation oncology. The doctors’ knowledge and experience, available published studies, and facilities accessibility in the treatment-center/hospital/clinic guide the decisional process. Expertise and opinions converge to form, in a collective decisional effort, the optimal treatment. While the combined clinical and empirical knowledge of tumor board members yields improved outcomes, the decision-making process is often imprecise, particularly when a patient’s status does not match cohorts in prior clinical investigations. Furthermore, many physicians do not have access to the multidisciplinary expertise of a tumor board.

With the growing amount of data collected for individual patients and cancer populations, a general and robust mathematical framework may contribute to a reproducible clinical decision using a reliable decisional algorithm. Ideally, such an algorithm would systematically and rigorously integrate patient-specific data with published cohort studies and large-scale population data from multiple institutions to predict treatment response with potentially adverse effects from all available clinical options. Such algorithms are not built to replace the oncologists and medical expertise; instead, they are proposed to help integrate and rigorously analyze the ever-increasing amount of data on highly heterogeneous diseases with significant inter- and intra-person heterogeneity for informed clinical decision making.

Historical examples in this directions are available already since late ‘80s 1,2. Nowadays, Artificial intelligence (AI) is used in evidence-based learning to support the decision-making process 3. The most notable example of this approach is probably the IBM Watson Health Program 4, even though less complicated online applications based on statistical indicators non/inclusive of past or modern genomic tests (e.g., Oncotype DX, but see also Mamma/BluePrint) such as Adjuvant! or PREDICT became available much earlier 5-7, not without skepticism 8. However, mechanistic mathematical modeling akin to clinical decision-making involves many degrees of freedom, or variables and parameters, such that models with different biological assumptions can simulate the selfsame datasets. When applied to patient-specific clinical data, this assortment of models and model predictions complicates decision making.

Embodiments of the present disclosure exploit Bayesian statistics to provide well-developed principles and frameworks to recapitulate the tumor board decisional process in terms of probability. The tumor board discussions can be formalized as an optimization process, acting on a suitably defined fitness function for the patient. Here, a flexible decisional framework inclusive of several clinical solutions from both the literature and the clinical tumor board expertise is presented such that, by having a fully comprehensive view on the possibilities of outcomes of cancer therapy, i.e., a “panoptic view” on the problem, it can attempt to rank available solutions by the likelihood of success, and therefore to suggest a “best” one within the uncertainties.

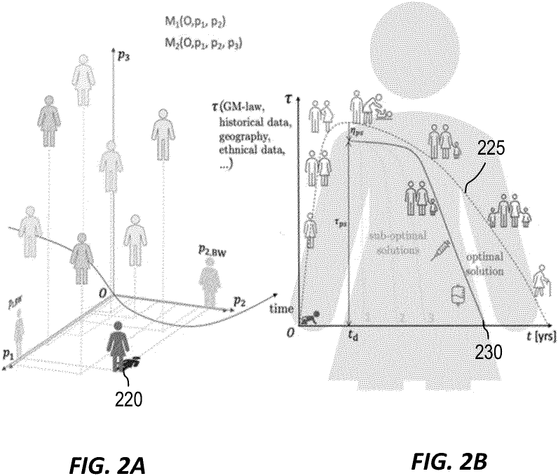

In this approach, each patient is a set of clinical data points in multidimensional parameter space, including demographics, clinical diagnosis, laboratory values, histologic features, comorbid conditions, current medications, and so on upon which the best (combination of) therapies need to be identified. Referring now to FIGS. 2A and 2B, schematic diagrams depicting an example model are provided. As illustrated, every patient of a trial is located in a point in the space of parameters of the model considered. For example, the patient under consideration, a blue woman (BW) 220, has coordinates {p₁=p₁,BW, p₂=p₂,BW} in model M₁(O,p₁, p₂), coordinates {p₁=p₁,BW, p₂=p₂,BW, p₂,BW=0} in model M₂(O,p₁, p₂, p₃). The common origin O of the defining set of parameters travels on the timeline; dynamical systems of equations are considered, hence the only parameter common to all the models is the time t (here represented ideally with a black curved line with a direction passing through O). In FIG. 2B, each patient the BW specific life expectancy function is considered, τ=τ(t). The Gompertz-Makeham law of mortality (GM-law, black dashed line 225) is sketched. At t_{d}, the BW receives the diagnosis of cancer. A negative slope for τ at t_{d} is assumed, and it is further assumed that the patient-specific life expectation τₚₛ(t_{d}) to be penalized under the GM-law by a penalizing factor ηₚₛ because of geographic, ethnic, or social factors, e.g., the BW patient is a former smoker. The red curve 230 is the optimal trajectory of life expectancy as a function of time for a patient predicted by the optimal patient-specific treatment identified by the tumor board. The sub-optimal trajectories, i.e., the temporal evolution of a decisional curve, e.g., yellow-dashed curves labeled 1, 2, or 3, live below the optimal path with curve 3 to be preferred over 2 and 2 to be preferred over 1 because its intercept with the y-axis (i.e., the death of the individual) is farther on the right (i.e., the life is longer). The evolutionary tumor board is the taskforce act to choose the optimal red curve.

The model examines available treatments, including surgeries, radiotherapy, chemotherapy, immunotherapy, or psychological support in the context of the desired outcome (tumor control, palliation, etc.). For example, the model formulates the probability that a treatment X1 or perhaps a series of treatments X1 combined with X2, X3, ..., will produce some outcome Y, e.g., the tumor burden, relapse-free survival, tumor control for 12+ months, and similar. Mathematically and clinically, both the existence and uniqueness of a successful solution are not always available, and the tumor board needs to identify suboptimal solutions. In FIGS. 2A and 2B, the optimal solution shifts the life expectation line intercept with the time axis to the right as much as possible with additional output on that prolonged life quality.

### Bayesian Decision Making

The attempt to model decisional processes starting from logic deductions finds its natural setting in the Bayesian framework⁹. In some embodiments, S may refer to a clinical hypothesis of interest (e.g., S =“radiotherapy can control tumor burden”, or the S=“drug X will increase time to progression compared to drug Y”) and I may refer to as the proposition representing prior/previously-acquired information (e.g., I = “the tumor is an early-stage breast cancer without lymph node or distant metastases”). The plausibility of the sentence S given (conditional on) the truth of the information I is called prior probability and labeled as Pr(S|I). What is the patient-specific likelihood that the tumor burden is controlled, e.g., by radiation therapy? The patient-specific probability is obtained once patient-specific data D are acquired (e.g., D =“the patient tumor is 3 cm in diameter”), and this probability is labeled as Pr(D|S,I). Then, the posterior probability of interest, i.e., the probability that the tumor burden can be controlled by radiotherapy provided that the tumor is early-stage and positive for a molecular biomarker, is given by the Bayes’ theorem 9:

$$Pr \left(\left(S\right| D , I\right) = \frac{Pr \left(\left(S\right| I\right) Pr \left(D \left|S , I\right)\right)}{Pr \left(D \left|I\right)\right)} ,$$

where Pr(D|I) is the normalization constant.

To identify the treatment with the highest likelihood of success requires the ability to grade different treatment models, frequently dealing with non-gaussian/skewed error likelihood, that can interpret the same data rigorously in a patient-specific way. Inherent to the Bayesian framework is a natural way to rank diverse model solutions. Here, Bayesian decision making on this “model of models” for oncological decision theory is discussed.

Bayes’ theorem, Eq. (1), encodes previous knowledge that can influence an outcome. In the Bayesian interpretation, the probability is a (real number) measure of a proposition/hypothesis plausibility given the truth of the patient-specific information acquired. Most textbooks on Bayesian statistics introduce a comparison between models. The interested reader is referred to the many excellent extended reviews on this topic ¹⁰⁻¹².

It is assumed that a set of models is available to work in synergy to achieve the optimization problem introduced above where different treatments represent the set of models, e.g., a combination of different chemotherapeutics, surgical procedures, radiation therapies, or immunotherapies. For simplicity, it can be assumed that the different treatments are all available in the hospital. A patient can be identified as a point in a multidimensional space, p = {p₁,..., p_{N}}, where each value p₁, p₂,. .., p_{N} is some clinically available measurement (e.g., age, gender, tumor burden size, Prostate-Specific Antigen level (PSA), white blood cell count, etc.) at the time of the diagnosis t = t_{d} (see FIGS. 2A and 2B). The decision support framework must then establish each variable’s role, according to its clinical significance for the cancer treatment, based on historical literature and other available clinical trial outcomes. For example, in prostate cancer treatment, metastatic sites and initial Gleason scores are relevant, but the gender is fixed, and specific blood cell counts are probably not prognostic unless abnormal. Once the treatment response model is identified, patient-specific disease trajectories can be simulated to optimize and adapt therapy following the model forecasting ^{13(p20219)}. Such a framework would then have to dynamically analyze and switch between different solutions (i.e., treatment approaches or protocols) when new data (such as clinical response measurements) or treatments become available.

### Bayesian Oncology Model of Models (MoM)

All the models may be compared in some output, i.e., in some clinically relevant metric such as a tumor marker (e.g., PSA), or the tumor volume Vᵢ, or survival τᵢ. Different therapies are then scored on that scale, e.g., the effects of radiation therapy or chemical or immunological treatment with or without surgery on overall survival. Thus, highly different therapeutic strategies can be fit into the same patient-specific set of data.

An essential ability of a probabilistic descriptive/predictive framework is its ability to deal with continuous (e.g., PSA values) and discrete variables (e.g., disease-free or disease progressed). Furthermore, since virtually all clinical data are collected at discrete intervals (e.g., CT scan every three months), the model accommodates discontinuous neoplasia volume reduction or, as in surgical resection, with simple step-functions. Finally, if each model is considered with its prior distribution over a joint likelihood, the model with the highest probability (global-likelihood/evidence) naturally leads to model selection.

For each model considered Mᵢ, the prior state of knowledge, I, is encoded into a prior probability distribution Pr(pᵢ|I), with pᵢ = {p₁,..., pₙ}ᵢ being the set of parameters for the model Mᵢ, The first step is to establish a prior distribution of credibility for the i^{th}-model parameter values pᵢ. By training, validating, and testing over many clinical cases, a library of examples is built that shape the prior distribution and increase the MoM prediction efficiency. In this way, when new drugs/techniques/data become available, they can be added to the library of models or model parameters, reshaping priors’ predictive power (after eventual retraining of MoM). For illustration purposes, little or null prior knowledge of the success rate that a specific model (i.e., treatment; model Mᵢ ), has on a particular type of cancer can be assumed. Thus, a model descriptive of a brand-new drug or illustrative of a new theoretical framework to explain the disease and weak knowledge of the parameter value has a broad probability that would span a wide range of the parameter space (FIG. 2A).

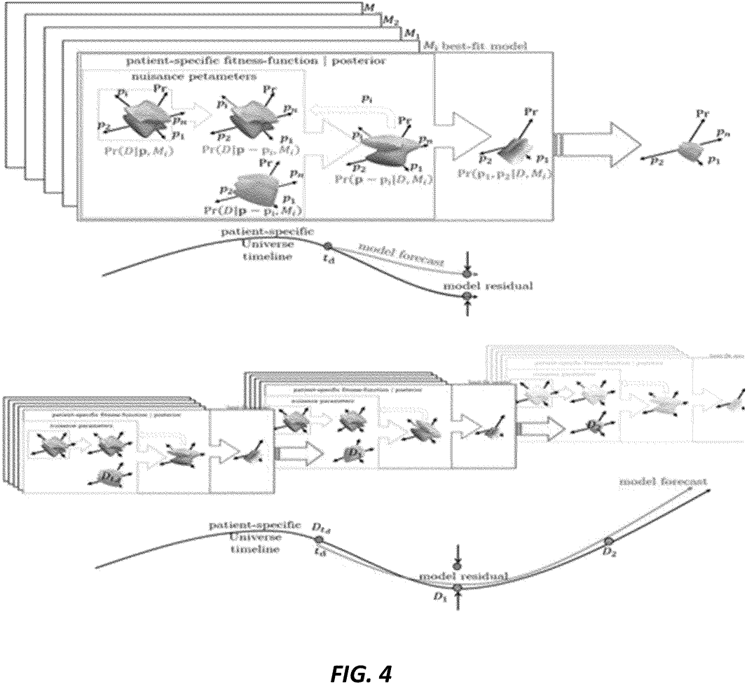

Referring now to FIG. 4, a schematic logic-flow of a clinical treatment augmented by the MoM framework is depicted. MoM is offered as a cloud computing service: it does not store patient generalities; instead, it outputs the optimal solution available to an inputted patient data points under discussion in a tumor board. The same service can be made accessible to a qualified medical doctor as a consultation-dynamical-library of treatment outcomes. Afterward, they might eventually defer the patient to a clinical structure where the suggested treatment is available.

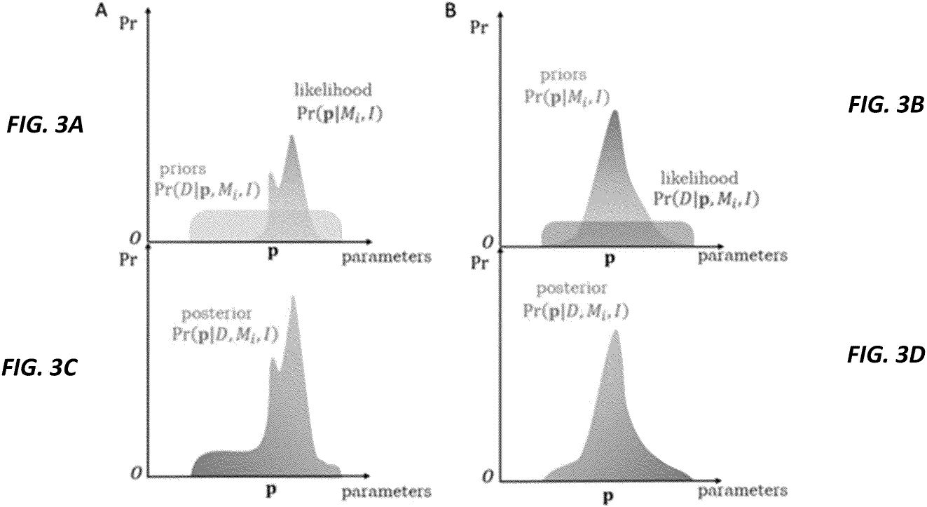

The Bayesian analysis provides a precise redistribution of the probability over the model parameter range once data, say the j^{th}-dataset considered Dⱼ, becomes available through the likelihood terms Pr(Dⱼ|pᵢ,I). In the assumption of identical and independent distributed errors for Dⱼ, the central limit theorem 10 or the maximum entropy principle 9 can be advocated to combine testable information I, with Shannon’s entropy (or Shannon-Jaynes, or Kullback entropies) and measure the uncertainty in a unique posterior distribution function through the use of the likelihood Lᵢⱼ(I) = Pr(Dⱼ|pᵢ,I). Under quite a broad general hypothesis, these principles assert that unless some information justifies the use of other sampling distributions, Gaussian likelihood for the error distribution makes the fewest assumptions possible about unavailable information on the collected data. Hence, this approach yields the most conservative estimate because no model is assumed a priori to be better than another. Instead, all models Mᵢ are considered correct, and each single data value d is related to a model value m, through an error e which represents the unknown “error counterpart” in the measurement of the data d. Here, a Gaussian distribution describes the source of errors (a noise with finite variance) for the error e. A new patient at the beginning of treatment, i.e., with a few data constraining his/her treatment/model, can be encoded with much fewer specificities, i.e., an extremely broad likelihood (FIG. 3B).

Once the posterior is obtained from the previous two steps for each of the models Mᵢ, i = 1,..,n_{N}, with N = N(t) the number of models considered (not necessarily constant), the patient-specific-fitness function

$$Pr \left({\text{p}}_{i} \left|D , I\right)\right) = \frac{Pr \left({\text{p}}_{i} \left|I\right)\right) Pr \left(D \left|{\text{p}}_{i} , I\right)\right)}{Pr \left(D \left|I\right)\right)} ∀ i ,$$

needs to be maximized. The topology of a nonlinear model posterior can be very intricate with many hills and valleys. Fortunately, the past 20 years have seen considerable advancement in algorithms to perform Bayesian calculations though there is no general solution available to the global optimization problems ^{10,11,14,15}. Roughly speaking, the most common search approach is based on asymptotic normal (i.e., truncated) approximations for a small number of parameters as the Bayesian Information Criterion (BIC) approximation to the log-marginal Pr(D|pᵢ,I), or on the Laplace approximation to the posterior Pr(pᵢ|D,I) around the mode, together with more numerical approaches based on random search techniques as Monte Carlo, Simulated annealing, genetic algorithms for a more substantial number of parameters, or a combination of the above. In most cases, each model is built upon a large number of parameters with only a subset being of interest for clinical decision making or, likely more prominently, several parameters that have to be included cannot be validated by data. Still, these parameters must be quantitatively accounted for without knowing their hidden probability distribution function because of their influence on fitting the model to the available data. These so-called nuisance parameters can be integrated out or marginalized.

As introduced above, a beneficial aspect of Bayesian statistics is the ability to efficiently couple discrete and continuous variables. This feature stands at the basis of the model comparison. When two different models explain the same dataset equally well (e.g., by producing comparable χ² values or comparable log-evidence in a fitting procedure ^{16,17}), a rigorous and reproducible approach needs to select one model, or treatment, over the other. The possibility of labeling the models lets us consider the model index itself as an independent parameter. The selection process hence results from an inference problem on the (discrete) model number. For example, the celebrated odd ratio of the probability of M₁ over M₂ simplifies as

$${O}_{12} = \frac{Pr \left({\text{p}}_{1} \left|I\right)\right) Pr \left(D \left|{\text{p}}_{1} , I\right)\right)}{Pr \left({\text{p}}_{2} \left|I\right)\right) Pr \left(D \left|{\text{p}}_{2} , I\right)\right)} = \frac{Pr \left({\text{p}}_{1} \left|I\right)\right)}{Pr \left({\text{p}}_{2} \left|I\right)\right)} {B}_{12} ,$$

with B₁₂ the Bayes factor of model 1 over model 2. Note that the model-index probability is sensitive to the entire parameter space, not only to the single model’s prior distribution at its best-fitting parameters position. More peaked prior distribution on well-fitting data will result in a higher probability density function (PDF) and, vice versa, when the prior distribution of a model flattens the PDF over a more extensive parameter range that does not fit the data well, the posterior PDF will tend to be small. This characteristic is advantageous in the MoM approach, where models of different complexities may be simultaneously considered. The more complex models will always be able to fit data better than restricted models. MoM balances data fit and model complexity (i.e., degrees of freedom provided by the number of parameters) and can select simpler models with fewer degrees of freedom over more complex models. By diluting the prior probability over larger areas, the more complex model assigns a lower chance for any parameter value that fits the data, resulting in a downweighed PDF. However, a more complex model will be selected if parameter values and model dynamics that are not accessible by a more restricted model provide a sufficiently better fit to the data.

If new data or a full new dataset Dⱼ₊₁ is acquired (e.g., a new value of a biomarker for the patient or clinical trial results published elsewhere) MoM does not need to be (re)trained, including the original data set. There may be obvious situations where retraining is unavoidable, e.g., because some clinical options are not available because of geographical constraints, economical constraints, psychological constraints, availability of a new vaccine etc. Bayes’ theorem is applied to compute each model’s new posterior to redistribute the latest knowledge state. The most recent prior, I′, is the posterior derived from Dⱼ, I, i.e., I′ = Dⱼ, I. Then, the new posterior is

$$Pr \left({\text{p}}_{i} \left|{D}_{j + 1} ,\right) {I}^{′}\right) ∞ Pr \left({\text{p}}_{i} \left|{I}^{′}\right)\right) Pr \left({D}_{j + 1} \left|{\text{p}}_{i} , {I}^{′}\right)\right) .$$

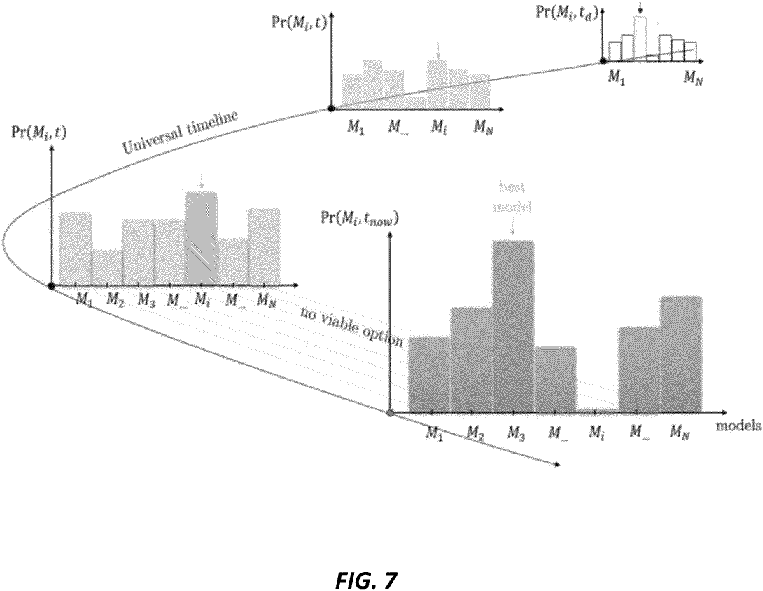

This iterative process can shift the weights, thus optimal selection, from a model to another (FIG. 7). Vice versa, the inclusion of a significant new paradigm, treatment, or approach (e.g., the discovery of a vaccine) might, of course, have such an impact to require MoM retraining to include availability factors (e.g., distribution factors, geographic factors).

Referring now to FIG. 7, a schematic diagram depicting a Posterior distribution function (PDF) of the MoM over the model index is provided. The models have indexed Mᵢ for i=1,..., N, over time and graded depending on their PDF. They gain or lose weight over time, depending on how new pieces of evidence are acquired. For example, at the time of the diagnosis t_{d}, the more suitable option could be surgery. After surgery, it can follow radiotherapy, a cycle of chemotherapy, etc., but the option of a second surgery, e.g., a second prostatectomy (the removal of partial or total part of the prostate), is less or no more viable. Error bars on the evidence histograms, despite available with classical technologies e.g., nested-sampling (Skilling 2004), are omitted for the sake of simplicity in this context.

Finally, superseding the global optimization problem mentioned above, a priori, MoM is not expected to provide a usable or meaningful full answer to the therapy selection problem. The approach proposed is a data-driven approach, both in the use of the priors, built on literature/trial results and in patient-specific data. Data are provided with errors that inevitably propagate on the model selection process. The global-likelihood/evidence of models M₁ and M₂ are determined at the best of uncertainties that impact the Bayes factor B₁₂ determination. Therefore, MoM might determine the best clinical path to follow but not outside any reasonable doubt, i.e., not outside the errors due to the available data quality. For this reason, the instrument proposed to introduce in this oncological contest (the tumor board) should be considered as a suggestion in the hands, and under the control, of the medical oncologist in charge.

### Model of Models Decision Making

Before presenting an example of decision-theory applied to tumor forecasting, the mathematical mechanism that leads to the decision is addressed. The principle-of-operation of this mechanism with the help of an example is detailed below, i.e., a hypothetical situation where the Bayesian decisional theory introduced above is crucial in helping a tumor board deciding which clinical path to follow. Despite the case being elementary and meant to match a situation with a well-known decisional output, it is pedagogical in its attempt to show how a tumor board opinion is mathematically coded and treated in the present formalism. The example is inspired by the Laplace approximation mentioned above. Still, it does not require the use of Gaussian approximation or the knowledge of information theory. Instead, it focuses on transmitting how the decisional process happens, i.e., it proposes an exemplification of the MoM model selection.

### Model Forecasting and Decision Theory

One of the statistical analysis’ aim is undoubtedly to aid a decision process when data analysis has suggested the best model available outside any reasonable doubt. It may be implicitly assumed that a goal is to estimate the probability of medical treatment to prevent or delay disease progression and death.

Leading Health Informatics and Medical Informatics Journals cover the Bayesian approach to model forecasting (e.g., Journal of the American Medical Informatics Association, Journal of Medical Internet Research, Medical Decision Making) together with numerous bestseller books ^{14,15,18-20}. Nevertheless, autoregressive moving average ²¹, vector autoregressive (VAR) models ²², together with the broad class of regressive neural network ²³, e.g., the long-short time memory ²⁴, are mainly focused on predicting the future data of a time series from the sequence of data collected rather than from a model comprehensive of the biological mechanisms involved as assumed for MoM.

In accordance with the present disclosure, a framework is presented that is able to engage the cancer description over its biological multiscale, robust in forecasting the evolution of the disease, and readily available to provide a biological interpretation of the results. As detailed below, and as illustrated in FIG. 7, the basics of such methods are reviewed by extending the context of the example provided above with simple Bayesian considerations, but focusing on the forecasting problem, whence medical decisions depend on data gathered after the first decision at t_{d} has already been taken.

### Tumor Board Evolution: Model of Models Decision Process

Classically from the posterior probability of the best model, the expectation value E[*] of a suitable defined function can be determined as the life expectancy τ from the evidenced best model at the medical screening time, conditional to the decision d, E[τ|d]. With additional data and tumor dynamics becoming available from a patient on the clinical response to therapy, MoM exploits its flexibility to relocate the probability over the entire parameter space, including the model index, to evaluate treatment adaptations. If a patient does not respond to a drug or drug change, the new information provides new prior data to recalculate posteriors for the remaining treatment options. The MoM approach automatically proceeds to this modal PDF’s reallocation, suggesting (when existing) the best combination of treatments available within the errors (see FIG. 7).

In the section above and the dedicated clinical in Example 1, where and how the decisional process happens is deeper explored. In Example 2, a more classical result of the Bayesian framework is presented: the frame’s forecasting character and its ability to include new information. Again, as Example 1, the concepts are developed elaborating over an example of clinical interest. Many self-similar exercises are available in the literature or on the web, especially concerning Medical research.

The idea of a framework to support the decisional theory modeled on tumor boards’ function to identify patient-specific clinical pathways is presented herein. Cancers are highly heterogeneous diseases with significant inter- and intra-person heterogeneity and high variability in clinical categorizations, definitions and delivery of treatments, and outcome determination. For any clinical decision-making, it is essential to rely on medical doctor experiences and the most accurate data and account for uncertainty and probabilities - for which Bayesian approaches are strongly suited. The proposed models of Models (MoM) is a fully comprehensive ecosystem able to account for patient-specific data, data uncertainty, different data-driven biological models, various treatment approaches, and, most importantly, it is a way to include human expertise represented by the tumor board. MoM’s strength is its panoptic view on the different aspects contributing to optimal therapies with reproducible uncertainties and confidence measurements. Exploiting the coexistence of opinions, equations, techniques resulting from diverse expertise (chemistry, physics, and biology), MoM aims to offer a reproducible framework to compare and upgrade knowledge on cancer therapy.

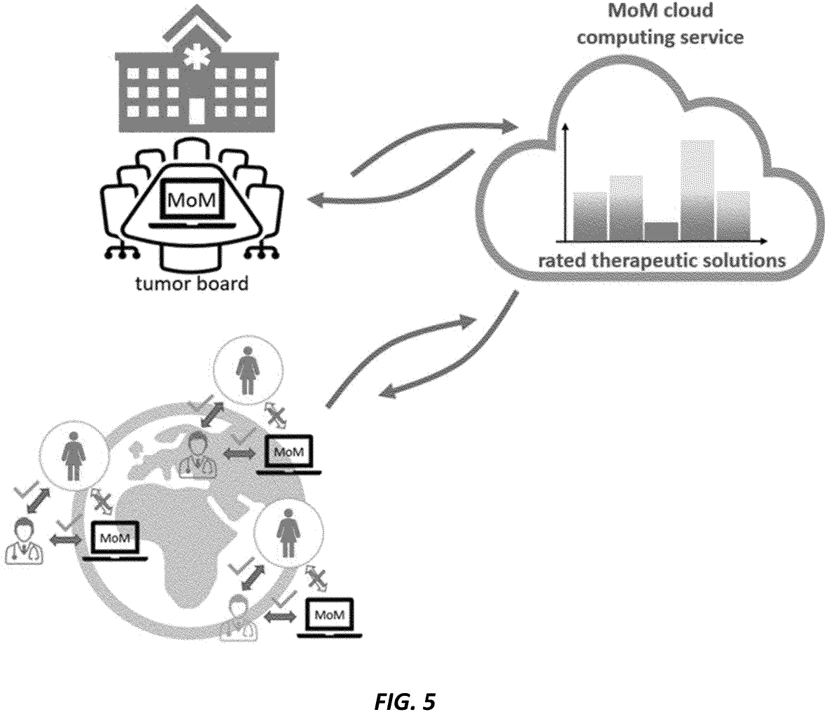

Despite being transparent to the clinician’s perspective, MoM is intended to be a freely accessible library of posterior probabilities of already-actioned (both successfully and unsuccessfully) clinical path on specific tumors. Any tumor board, or clinician-oncologist, might want to access it, e.g., through a webpage. Once the clinical parameter of a patient-specific case of interest is inserted, MoM will rate the relative merits of the therapeutics paths. One of the strengths of the MoM approach is that the largest the number of points is in the database (or inputted by oncologists spread worldwide), the more useful and efficient this instrument turns out to be, not only in a tumor board setting (i.e., in an in-person meeting) but also, out-scoping, as rapid informative clinician instrument. Nevertheless, this very same approach might also hide a weakness. If MoM builds up its prior on a larger and larger database, its response might be closer and closer to the optimal clinical pathways to follow. Influential priors might be extremely sensitive to the loco-regional constraints: many therapeutic solutions immediately available in large cities might vice-versa require patients living in smaller towns to face long trips that might not be possible because of their clinical conditions. Evaluation of software design that might include retraining options may be necessary to make MoM useful in certain applications. A workflow of the MoM concept can be evicted from FIG. 5.

Referring now to FIG. 5, a schematic logic-flow of a clinical treatment augmented by the MoM framework is provided. As depicted, MoM is offered as a cloud computing service: it does not store patient generalities; instead, it outputs the optimal solution available to an inputted patient data points under discussion in a tumor board. The same service can be made accessible to a qualified medical doctor as a consultation-dynamical-library of treatment outcomes. Afterward, they might eventually defer the patient to a clinical structure where the suggested treatment is available.

Note how MoM is a logic-probabilistic tool for informative purposes only. It is not intended by any means to indicate the treatment pathway: in its only intended to rate the therapeutic options whose selection is left first and only to the patients through their medical doctors (MDs). This concept is represented in the figure as a connection between MoM patients passing throughout the MDs. Furthermore, MoM’s development as a computational tool (e.g., in a cloud computing service) instead of a patient database is aimed to stress that no patient generalities need to be stored and only their correspondent data-point needs to be inputted.

In conclusion, the MoM framework is conceptually designed to identify optimal treatments based on patient-specific data-points in the context of information from published studies and the institutional (or multi-institutional) databases. Here, MoM’s concept is presented as a decision support tool and provide the initial clinical translation step. To be utilized in tumor boards and by individual oncologists, it must be trained, tested, and prospectively validated for specific cancers and purposely developed mathematical and statistical models of cancer progression and treatment response. Once implemented, the Bayesian MoM approach will have to be compared with other decision support tools to evaluate its clinical applicability and to ethically integrate the framework into clinical practice so that no harm is done.

### Example 1: Example of Bayesian Model of Models For a Hypothetical Patient

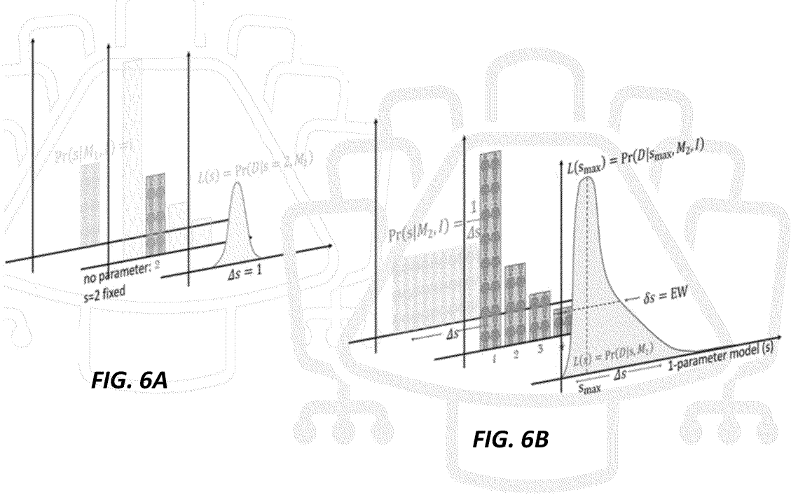

With reference to FIG. 6, two models (i.e., two treatment options) proposed for a 65-year-old woman with operable early-stage (stage II) breast tumor, hormone-receptor negative, human epidermal growth factor receptor 2 (HER2) positive, and axillary node-negative are compared. For demonstration purposes, it is assumed that the virtual patient has no preference for treatment options. However, individual patient preferences are straightforward to include in the MoM framework by adjusting the Bayesian priors. In the demonstrative example herein, it is assumed that all discussed treatment models align with the treatment guidelines for this specific cancer type. Furthermore, to achieve interesting clinical results, the posterior probability must be augmented, encoding literature’s wealth on the particular case: For example, it might be worth empowering the results considering the Early Breast Cancer Trialists’ Collaborative Group (EBCTCG) works ²⁵⁻³⁰. Here, this part of the process is omitted that otherwise would completely occult this exercise’s goal.

For prospective translation, evidence-based guidelines must interface with the Bayesian MoM to correct each treatment’s prior distributions. Suppose the considered patients do not favor one treatment. In that case, the model is not incorporated, or the prior distribution is defined such that the posterior probability of model selection reflects clinical standard.

A first model proposed in tumor board, Model-1 (M₁), offers the treatment to be mastectomy followed by radiotherapy, i.e., no free parameters. This model is compared with the opinion of a second model (M₂) that proposes the use of radiation (e.g., after the surgery) and four alternative surgical procedures offered by the surgeon. Therefore, this second model introduces one parameter, the parameter “surgery” s, ranging from 1 to 4 s = {1,2,3,4}. For s = 1 the surgery is a lumpectomy (partial mastectomy), s = 2 a mastectomy, s = 3 a mastectomy with implant placement, and s = 4 all the other less attractive procedures are lumped together 31. The PDF can be sketched as depicted in FIG. 6. Therefore, the therapeutic model M₂ includes M₁ as particular case: s = 2, the mastectomy, with its rate of success and risks exactly as in M₁, with the difference that in M₁ it was given as the unique option available without choice (M₁ has no free parameter indeed). M₂ is first analyzed as being M₁ a particular case of M₂, i.e., they are nested models (FIG. 6).

As depicted in FIGS. 6A and 6B, the two models proposed by two different tumor boards are compared in their two different outcomes. The right (blue) tumor board (FIG. 6B) consider the rate of success of a treatment, i.e., a probability density function (pdf), as a function of a patient-tailorable parameter, i.e., the type of surgery, s, with s=1 lumpectomy (partial mastectomy), s=2 a mastectomy, s=3 a mastectomy and implant placement and with s=4. The prior is assumed to be not informative for simplicity; therefore, here is represented as a flat grey histogram at the bottom of the figure’s right because not of interest. Vice versa, the informed opinion of the tumor board (e.g., from a surgeon who preventively visited the patient) is sharply peaked in favor of option s=1. This is visualized as a series of four histograms in the middle. The histogram of a continuous probability density function is the curve corresponding to the histogram distribution. From the continuous curve, the EW of interest can be determined. Analogous is the treatment for the left (green) tumor board (FIG. 6A) where, for the considered example, it is assumed that the only option offered to a patient is a mastectomy (therefore no free parameters in the model/tumor board opinion), and it corresponds to one of the options provided by the blue-tumor board. Hence, a case of nested models is presented. This simplifies the arguments: the prior is unitary (last left graph), the green pdf in FIG. 6A equals the blue tumor board pdf for the value s=2 (and in the center panel, the corresponding histograms of the blue pdf are sketched in grey to guide the reader view), and the continuous pdf is just drawn in the first of the green plots as a purely indicative guide. The green area under the pdf is smaller than the blue area and thus EW<Δs graphically depicts the equations in the text.

Patient demographics and pathology data increase information. Therefore, it can be assumed that the likelihood function L(s) = Pr(D|s,M₂,I) is more peaked than the prior Pr(s|M₂,I). This means that the standard-of-care approach (given by a combination of surgery and radiation) that blindly might be hypothesized without patient-specific information (as in Model 1) will be personalized by considering the patient-specific information encoded in the patient likelihood. Assuming a flat prior

$$1 = \int {}_{Δ s} d s Pr \left(s \left|{M}_{2} , I\right)\right) = Pr \left(s \left|{M}_{2} , I\right)\right) Δ s ,$$

from which it is understood that

$$Pr \left(s \left|{M}_{2} , I\right)\right) = \frac{1}{\text{Δ} s} .$$

Then, it is always possible to define a characteristic width δs (called the equivalent width EW) implicitly so that for the likelihood of M₂ the following relation must hold:

$$\int {}_{Δ s} d s Pr \left(D \left|s , {M}_{2} , I\right)\right) = Pr \left(D \left|{s}_{max} , {M}_{2} , I\right)\right) ⋅ δ s ,$$

with an evident maximum at the parameter s = sₘₐₓ = 1, i.e., a lumpectomy (breast-conserving therapy, BTC) followed by radiation therapy. Note how, in this way, the integral, i.e., the blue area under the curve in the figure, is therefore obtained as a product of the basis EW = δs times the maximum height of the curve Pr(D|s_{max,} M₂, I). Therefore, the global-likelihood of model 2, L = L(M₂), reads:

$$\begin{matrix}L \left({M}_{2}\right) = Pr \left(D , {M}_{2} , I\right) \\ = \int d s Pr \left(s \left|{M}_{2} , I\right)\right) Pr \left(D \left|s , {M}_{2} , I\right)\right) \\ = \frac{1}{Δ s} \int d s Pr \left(D \left|s , {M}_{2} , I\right)\right) \\ ≅ Pr \left(D \left|{s}_{max} , {M}_{2} , I\right)\right) \frac{δ s}{Δ s} .\end{matrix}$$

Hence, approximatively:

$$L \left({M}_{2}\right) = L \left({s}_{max}\right) \frac{δ s}{Δ s} .$$

In the case of the model M₁, no free parameter is assumed to be tuned on the patient-specific case and L(M₁) is simply the likelihood of M₂ at s = ŝ (s = 2 in the example pictured in FIGS. 6A and 6B) because, for simplicity, nested models are assumed. Therefore

$$Pr \left(D \left|{M}_{2} , I\right)\right) = Pr \left(D , \hat{s} , {M}_{1} , I\right) = L \left(\hat{s}\right) .$$

Now all the instruments to understand when the framework effectively favors model 2 over model 1 are provided. The Bayes factor of Eq. (3), here rewritten to output the model 2 over model 1 preference, approximates as:

$${B}_{21} \approx \frac{Pr \left(D \left|{s}_{max} , {M}_{2} , I\right)\right)}{Pr \left(D \left|\hat{s} , {M}_{1} , I\right)\right)} = \frac{L \left({s}_{max}\right)}{L \left(\hat{s}\right)} \frac{δ s}{Δ s} .$$

As evident, the likelihood ration is hardly in favor of the simpler model (the mastectomy) because M₁ contains M₂ as a particular case: Statistically, most women treated in the second facility have preferred option s = 1 (BCT and radiotherapy) indeed. Hence, the evidence introduced by the second surgeon opinion, and in the figure captured by the blue histogram, is higher for s = 1 than s = 2. s = 1 is indeed the max of the probability density function s = sₘₐₓ, therefore the option proposed by the first facility ŝ < sₘₐₓ. If the continuous approximation of the histogram distribution function is considered, the M₂ the likelihood in Eq. (7) is regarded as the area under the curve connecting the discrete histogram heights: a base δs that multiplied by the max height of the distribution L(sₘₐₓ) results in the area equivalent to the one under the curve. Nevertheless, the posterior width δs is narrower than the prior width Δs, and the second factor will penalize the complicated model M₂ for wasting parameter space ruled out by data. If the likelihood ratio is large enough to overcome this penalty, then Bayes’ factor will favor more complicated models.

If so far it is shown how the MoM mechanism favors the decision throughout the Bayes factor, a better sense of how important this mechanism is can be gained by adding to our consideration a systemic treatment (e.g., chemotherapy, hormonal therapy, or target therapy), i.e., adding a third model, M₃To the comparison. Because it is assumed to deal with a HER2+ case in a relatively early stage older woman, rather than considering chemotherapy or target therapy alone, a pharmacological cocktail is introduced as a discrete parameter, e.g., d = {1,2,3} where d = 1 is chemotherapy alone, d = 2 is a combination of chemotherapy and trastuzumab, and d = 3 the combination of chemotherapy, trastuzumab, and pertuzumab ^{32,33}. Again, the goal here is to quantitatively explore the Bayes factor influence in the decisional process’s mechanics rather than focusing on any real clinical aspect of the case under exam. The model M₃ has two parameters, one describing the surgery’s applicability and one the effectiveness of the systemic therapy. Because it is harder to sketch multidimensional space, a figure for M₃ PDF is omitted, but can be generalized as above. The above-identified parameters “s” and “d” are assigned a more flexible notation p₁ and p₂ in line with the previous sections, and the following is written:

$$\begin{matrix}Pr \left(D \left|{M}_{3} , I\right)\right) = \int {d}^{2} \text{p} Pr \left({p}_{1} \left|{M}_{3} , I\right)\right) Pr \left({p}_{2} \left|{M}_{3} , I\right)\right) Pr \left(D \left|{p}_{1} , {p}_{2} , {M}_{3} , I\right)\right) \\ = Pr \left(D \left|{\text{p}}_{max} ,\right) {M}_{3} , I\right) \frac{δ \text{p}}{Δ \text{p}} ,\end{matrix}$$

where

$$\frac{δ \text{p}}{\text{Δ} \text{p}} = \frac{δ {p}_{1}}{\text{Δ} {p}_{1}} \frac{δ {p}_{2}}{\text{Δ} {p}_{2}}$$

For example, it is assumed that

$$\frac{δ {p}_{1}}{Δ {p}_{1}} = \frac{1}{4} = 0.25$$

as can be evicted from the equivalent-width in the drawing of FIGS. 6A and 6B, where the histogram height at s = 1 is taken to be as high as the other three options taken together (after Jonczyk et al. 2011). Furthermore, because a similar PDF for the second parameter implies

$$\frac{δ {p}_{2}}{Δ {p}_{2}} < 1 ,$$

, it can be assumed, for example,

$$\frac{δ {p}_{2}}{Δ {p}_{2}} = \frac{1}{3} ≅ 0.33$$

(not shown in FIGS. 6A and 6B). Therefore, the penalty factor is as low as ~0.07, and the Bayes factor to favor M₃ given by the ration of the maximum likelihoods is

$$\frac{Pr \left(D \left|{\text{p}}_{max} , {M}_{3} , I\right)\right)}{Pr \left(D \left|{M}_{2} , I\right)\right)} = \frac{L \left({M}_{3}\right)}{L \left({M}_{2}\right)} \geq 1.3 \times {10}^{1} ,$$

i.e., more than ten times bigger: unless the data strongly argue for the use of systemic therapy, MoM would strongly argue in favor of radiotherapy and surgery alone.

Aside from the too simplistic approach³⁴, the merit of the example is merely highlighting the decisional process. If a surgeon comes to the tumor board with the idea of a type of surgery, this new information I brought from the doctor can be encoded. Bayesian decision theory is versatile enough to codify this surgeon’s opinion gained through personal experience and clinically collected data with the elementary likelihood distribution, as pictured in FIGS. 6A and 6B. Furthermore, when a new approach becomes available as in the case of Model 3, unless the success rate of the use of a chemical cocktail is statistically evident to be 13 times more successful, once all the information I is accounted for (e.g., extension, toxicity, etc.), there is no reason to proceed with too complex models.

### Example 2: Evolutionary Tumor Board for a Hypothetical Patient

Consider again the previous case of a 65-year-old woman with an operable early-stage (stage 2) breast tumor (say T2N0M0). From the best knowledge of the tumor board members, once supported by the framework developed here, the patient is best represented at the diagnosis by a model whose PDF allows to make only initially vague forecasts. Nevertheless, the tumor board just met the patient. Only a few data-specific clinical exams are available. Some information is still missing (e.g., luminal, HER2, and basal subtype), and the problems of overfitting in the complicated models led to an unstable solution.

In this case, these decision problems can be expressed as decision trees with accompanying uncertainties ³⁵⁻³⁸. In this multistage problem, the Bayesian inference is particularly useful in updating the state of knowledge with the information gained from additional tests and scans during therapy. Here, the concept of repeated tumor boards for individual patients is introduced (FIG. 7).

From the Center for Disease Control and Prevention (CDC) lifetables for 75-year-old females of all races and origins, the posterior with a life expectancy of 12.6 years (151 months) 39,40 with a 90% likelihood of no malignant tumor can be informed. Life tables implicitly contain competing risks to breast cancer mortality; however, explicit competing risks can be included in the analysis and only left out of this demonstrative example for simplicity. For demonstration purposes, it can be assumed that without any treatment, the life expectancy is about five months; in the case of radiotherapy, the life expectancy is 60 months; and in the case of surgery, her life expectancy is 90 months, with a 0.2% risk of death due to surgical complications 41.

The decisional path of the tumor board supported by a probabilistic framework proceeds as follows. The life expectancy τ, depending on the result of the biopsy test not yet obtained (e.g., on the cancer malignancy, subtypes, etc.), will result in the case the tumor board opting for no-treatment (say, model M₀). Then, the life expectancy without treatment τ0 is

$$\begin{matrix}{τ}_{0} = Pr \left(\cdot \right) {τ}_{\text{treat}}^{\text{no}} + Pr \left(◦\right) {τ}_{\text{tumor}}^{\text{no}} \\ = Pr \left(\cdot \right) ⋅ {τ}_{\text{treat}}^{\text{no}} + \left(1 - Pr \left(\cdot \right)\right) {τ}_{\text{tumor}}^{\text{no}} \\ = 0.9 ⋅ 5 \text{mos} + \left(1 - 0.9\right) 151 \text{mos} \\ = 19 \text{mos} ,\end{matrix}$$

where the presence of the tumor is denoted with “•” and the absence, i.e., not-presence, “¬ •” directly with “◦.” In the case of radiation therapy, model M₁, defines life expectancy τ1 by

$$\begin{matrix}{τ}_{1} = Pr \left(\cdot \right) ⋅ {τ}_{\text{treat}}^{\text{rad}} + Pr \left(°\right) {τ}_{\text{tumor}}^{\text{no}} \\ = 0.9 ⋅ 60 \text{mos} + \left(1 - 0.9\right) 151 \text{mos} \\ = 69 \text{mos} .\end{matrix}$$

Finally, the surgery option M₂ will lead to

$$\begin{matrix}{τ}_{2} = Pr \left(+\right) \left(Pr \left(\cdot \right) ⋅ {τ}_{\text{treat}}^{\text{surg}} + \text{Pr} \left(∘\right) {τ}_{\text{tumour}}^{\text{no}}\right) + Pr \left(-\right) ⋅ 0 \\ = \left(1 - Pr \left(-\right)\right) \left(Pr \left(\cdot \right) ⋅ {τ}_{\text{treat}}^{\text{surg}} + Pr \left(∘\right) {τ}_{\text{tumor}}^{\text{no}}\right) \\ = \left(1 - 0.002\right) \left(0.9 ⋅ 90 \text{mos} \text{+} \left(1 - 0.9\right) 151 \text{mos}\right) \\ = 96 \text{mos} ,\end{matrix}$$

where Pr(+) indicates the probability of surviving the surgery, and Pr(—) the chance of dying as a consequence of the operation 41. These calculations identify surgery as the clinical path that maximizes a patient’s life expectancy (τ2 > τ1 > τ0).

In the iteration of the tumor board (cf. FIG. 7) after new patient data (such as biopsy results) become available, classical Bayesian inference calculates the chance of the presence of cancer given the biopsy (Bio) result as:

$$\text{Pr} \left(\cdot \left|\text{Bio}\right)\right) = \frac{\text{Pr} \left(\cdot \right) \text{Pr} \left(\text{Bio} \left|\cdot \right)\right)}{\text{Pr} \left(\cdot \right) \text{Pr} \left(\text{Bio} \left|\cdot \right)\right) + \text{Pr} \left(∘\right) \text{Pr} \left(\text{Bio} \left|∘\right)\right)} ,$$

where the disease prior probability is the same as before Pr(•) = 0.9, the likelihood L for the model of biopsy performed is taken from the literature (e.g., considering the machine used or the technique used) and informs a probability Pr(Bio | •) = 0.21 that the biopsy detects cancer when it is effectively present and of a likelihood Pr(Bio|°) = 0.71 of a false positive (the biopsy claims cancer while it is no there)⁴². In this example:

$$\text{Pr} \left(\cdot \left|\text{Bio}\right)\right) = \frac{0.9 ⋅ 0.21}{0.9 ⋅ 0.21 + 0.1 ⋅ 0.71} = 0.72 ,$$

and the updated chance of the clinical path obtained, including these new priors in the previous model estimation, is hence: τ₀ = 6 mos, τ₁ = 46 mos, and τ₂ = 51 mos, respectively. If the biopsy is negative, i.e., no cancer is present, then

$$\text{Pr} \left(\cdot \left|\text{Bio}\right)\right) = \frac{\text{Pr} \left(\cdot \right) \text{Pr} \left(\text{Bio} \left|\cdot \right)\right)}{\text{Pr} \left(\cdot \right) \left(1 - \text{Pr} \left(\text{Bio} \left|\cdot \right)\right)\right) + \text{Pr} \left(∘\right) \left(1 - \text{Pr} \left(\text{Bio} \left|∘\right)\right)\right)} = 0.96 ,$$

and the updated expectancy is τ₀ = 108 mos, τ₁ = 161 mos and τ₂ = 66 mos, respectively. This result indicates that the new data acquired can change potentially the model to follow, i.e., from M₂ initially preferred with its 51 mos of life expectancy predicted to M₁ now offering 161 mos, thus shifting the clinical path from one model to another. Given the arbitrarily chosen life expectancies after therapies, the shift to radiation over surgery may indicate plausible values for benign disease.

The prostate is an exocrine gland of the male reproductive system dependent on androgens (testosterone and dihydrotestosterone) for development and maintenance. First-line therapy for prostate cancer includes androgen deprivation therapy (ADT), depriving both the normal and malignant prostate cells of androgens required for proliferation and survival. A significant problem with continuous ADT at the maximum tolerable dose is the insurgence of cancer cell resistance. In recent years, intermittent ADT has been proposed as an alternative to continuous ADT, limiting toxicities and delaying time-to-progression.

Several mathematical models with different biological resistance mechanisms have been considered to simulate intermittent ADT response dynamics. A comparison between 13 of these intermittent dynamical models and assess their ability to describe prostate-specific antigen (PSA) dynamics is presented. The models are calibrated to longitudinal PSA data from the Canadian Prospective Phase II Trial of intermittent ADT for locally advanced prostate cancer.

In accordance with embodiments of the present disclosure, Bayesian inference and model analysis over the models’ space of parameters on- and off-treatment are performed to determine each model’s strength and weakness in describing the patient-specific PSA dynamics. Additionally, a classical Bayesian model comparison on the models’ evidence is carried out to determine the models with the highest likelihood to simulate the clinically observed dynamics.

Embodiments of the present disclosure identify several models with critical abilities to disentangle between relapsing and not relapsing patients, together with parameter intervals where the critical points’ basin of attraction might be exploited for clinical purposes. Finally, within the Bayesian model comparison framework, the most compelling models in the description of the clinical data are described.

The prostate is an exocrine gland of most mammals’ male reproductive system. The normal prostate is dependent on androgens, specifically testosterone and 5α-dihydrotestosterone (DHT), for development and maintenance (Feldman and Feldman 2001). Prostate carcinoma (PCa) results from the abnormal growth of tissue from the prostate’s epithelial cells, which might induce metastasis in bones and lymph nodes. PCa is the second most common cancer in the US and the second leading cause of cancer-related death after lung cancer (Siegel et al., 2021). The average male age is 70 years of age at the time of diagnosis, with a strong asymmetry of the distribution biased towards older ages. PCa risk is often influenced by genetics. Men with a first-degree relative with PCa are twice as likely to develop it themselves; men with high blood pressure are also at higher risk of PCa. Treatment options typically include surgery, radiotherapy, high-intensity focused ultrasound, chemotherapy, and hormonal therapy.

Screening for PCa is commonly performed through rectal examination or the non-invasive blood biomarker prostate-specific antigen (PSA), although its efficiency remains controversial (Lin et al., 2008). Today, more robust marker indicators, such as the overexpression of prostate cancer gene 3 (PCA3) obtained from the messenger-RNA (mRNA) in the urines, are considered more suited to monitoring the cancer evolution (Bussemakers et al. 1999, p. 3; Laxman et al. 2008; Neves et al. 2008; Hessels and Schalken 2009, p. 3; Borros 2009; Qin et al. 2020). PSA is a measure of a hematic enzyme produced by the prostate. PSA levels between 4.0 to 6.5 µg L⁻¹are generally considered normal (with a strong dependence on age). PSA is naturally present in the serum, and usually, only a small amount of PSA of the prostate leaks into the blood. Hence high levels are an indication of prostatic hyperplasia or cancer. Since prostate cells and their malignant counterparts require androgen stimulation to grow, prostate cancer can be treated by androgen deprivation therapy (ADT), a type of hormone therapy. This therapy reduces androgen dependent (AD) cancer cells by preventing their growth and inducing cellular apoptosis.

Unfortunately, treating with ADT often results in a relapse in the form of hormone-refractory PCa due to the selection for the androgen-independent (AI) cells. Intermittent androgen deprivation (IAD) therapy, whereby treatment is cycled on and off, is often used as an alternative to ADT to delay treatment resistance. In IAD, androgen deprivation therapy is administered until a patient experiences a remission and then is withheld until the disease progresses up to a certain level. Clinical studies have shown that patients are responsive to multiple hormone therapy cycles, eventually delaying the androgen independence insurgence (Klotz et al. 1986; Larry Goldenberg et al. 1995; Bruchovsky et al. 2006).

Embodiments of the present disclosure consider models of intermittent therapy due to clinical interest and solve the inference problem using longitudinal PSA data from the Canadian Prospective Phase II Trial of IAD for locally advanced prostate cancer. This work aims to present the first systematic comparative study of IAD models, emphasizing their ability to disentangle relapsing and not relapsing patients and compare the models in the Bayesian framework. The goal is to detect the single model (or the group of models) that best represent the information in the considered dataset and, therefore, if possible, the most promising biological frame representing them. A general and historical review of the prostate cancer literature available models can be found elsewhere (Phan et al. 2020).

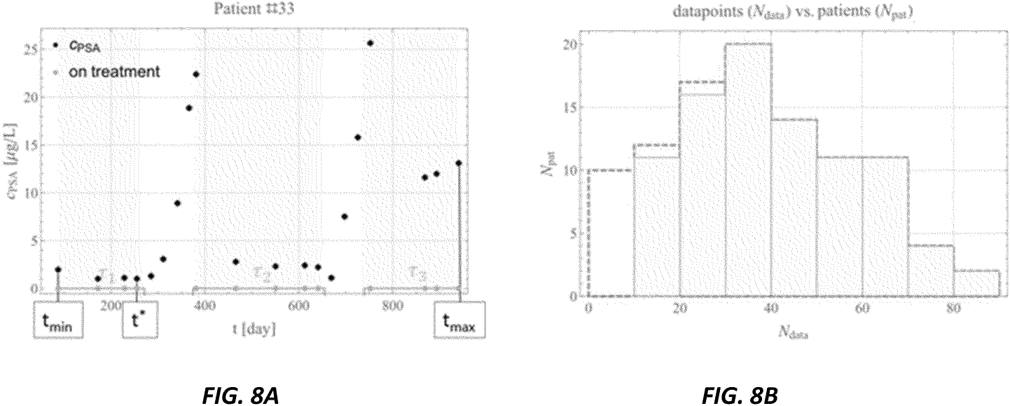

Data from the Canadian Prospective Phase II Trial of intermittent ADT for biochemically recurrent prostate cancer (Bruchovsky et al. 2006, 2008) is considered. The total patient number is Nₚₐₜ = 101. Their median pretreatment serum testosterone is 13.0 µg L⁻¹, ranging between 0.4 to 23.0 µg L⁻¹. Over a maximum of n = 5 intermittent ADT cycles, a median of 35.1 to 36.0 weeks is spent on-treatment (depending on n), and 25.6 to 53.7 weeks (e.g., n=5 and n=1 respectively), are off-treatment during the 6-year study. An example of a PSA profile for an individual patient is shown in FIG. 8A.

Referring now to FIGS. 8A and 8B, schematic diagrams depicting model data are provided. FIG. 8A illustrates PSA data for patient #33 from tₘᵢₙ=88 [day] to tₘₐₓ = 941 [day]. Black dots indicate PSA values (error bars are omitted due to little variability), orange points indicate where PSA was collected, and graphically represented as an orange continuous box function, evidenced only in this example panel by yellow shaded areas. FIG. 8B illustrates a distribution of the number of data points per patient. The original data is shown by the red dashed lines, while the selected subset of patients used in this analysis is shown in the yellow shaded region.

As depicted, patient #33 responded to treatment during the first two treatment cycles (τ₁ and τ₂) and progressed in his third cycle of treatment (τ₃). The oscillatory dynamics demonstrate the effect of the intermittent treatment, with a decrease in PSA during treatment and an increase once treatment is turned off. Each data point is assigned with an error of 1 day in time (i.e., the time resolution of the dataset) and a maximal PSA error value eₘₐₓ assigned of eₘₐₓ = 0.1 µg L⁻¹ assumed from the literature (Borros 2009).

The minimal PSA detection threshold is set as equal to Δ₁ = 0.1 µg L⁻¹, i.e., any patient data below this threshold is set to 0.1 µg L⁻¹. Patients with a minimal per-day fluctuation below 2.0 µg L⁻¹, i.e., a minimal per-day fluctuation of the KLK3 glycoprotein enzyme of PSA of a typical man (Morgentaler and Conners 2015), are excluded because such small fluctuations are considered natural and not pathological. To only consider PSA concentrations above Poisson-noise, patients with less than

$${Δ}_{2} = \sqrt{{N}_{\text{pat}}}$$

(i.e., the sample shot/Poisson noise) data points are also excluded. These exclusions result in the analysis considering data from 89 (Nₚₐₜ= 89). rather than 101 patients. The patients’ distribution per number of data points used after the selection process is shown in FIG. 8B compared to the original distribution.

The PSA trend shown in FIG. 8A is based on the interplay between two cellular populations, i.e., a compartment modeling approach. An androgen-dependent set of N_{D} ≥ 1 cell population (each with a concentration n_{D,k} = n_{D,k}(t), k = 1, ...,N_{D} representing the compartment concentration [µg L⁻¹], t ∈ ℝ time [day]) is assumed to contribute to the oscillatory behavior of PSA. Additionally, a set of time-dependent androgen-independent cellular populations of N_{I} ≥ 0 cell population n_{I,l} = n_{I,l}(t), l = 0, ..., N_{I}, also contributes to the PSA profile, such that PSA concentration c_{PSA} is given by c_{PSA} = f (n_{D,k},n_{I,l}), where f ∈ C⁰ is a function belonging to the class of continuous solution C⁰ (not necessarily smooth) of a suitably designed ODEs system. Any further dependence on space, temperature, and pressure is generally neglected in the IAD models’ compartment approach. Furthermore, f is often assumed to be a linear combination of the n_{D} and n_{I} compartments, e.g., c_{PSA} = Σₖ wₖn_{D,k} + Σₗ wₗn_{I,l} for some weights wₗ or wₖ.

By assuming ADT to be highly effective in the first treatment interval τ₁, n_{D}(t ∈ τ₁) ≅ 0 as is set as an initial condition (hereafter i.c.). This approach does not necessarily hold for τᵢ with i > 1: Generally, ∀i where c_{PSA} ≅ 0 can equally be assumed at this setting for the i.c. of the n_{D,k} = n_{D,k}(t) equations. Equivalently it can be assumed that a non-holonomic (i.e., with inequalities) condition for the fitting procedure holds at the beginning of the patient time series n_{D}(t) ≤ n_{I}(t) for some t ∈ τ₁. Furthermore, in most of the models that are accounted for, these considerations are articulated with the addition of a few extra equations that interpret, at a local or global level in the parameter space, the contribution to c_{PSA}(t) by the androgen quota, cellular plasticity, staminal cells populations, or other model specificities.

Finally, it is known from biological arguments that, under treatment, the models’ equations are designed to permit, at least for c_{PSA}, to tend asymptotically to the value c_{PSA} = 0. Any model that does not permit the phase state c_{PSA} = c_{PSA}(t) to reach approximatively null values for any t, i.e., t ∈ ℝ: c_{PSA}(t) ≅ 0, would fail to reproduce the patients whose first treatment is always successful (see FIG. 8A). Therefore, it is worth investigating if the models allow for stationary equilibria outside the treatment intervals and then if any of the patient best fit values have fallen close to those equilibria (when they exist). This behavior would imply a stationary or recurrent solution for the dynamics and, therefore, a constrained PSA’s evolution if this “basin of attraction” is achievable in a biological time of interest. This mathematical behavior does not imply that the patient can effectively reach the equilibria on the biological timescale of interest or a plausible point regarding toxicity levels.

The Bayesian regression approach stems from the concept of probability as a measure of the plausibility of a model given the truth of the information in the data presented above. First, the prior state of knowledge about the parameters considered is encoded p = {p₁, p₂,...} into a prior distribution function Pr(p|I), where I represents any available information. Typically, this can be achieved with a flat, uniform, and not informative prior at the beginning or with a sharper prior when the model is better trained. Secondly, the data set, D, is considered through the likelihood L(p,D) = Pr(D |p,I). Finally, the inference problem is solved, studying the probability distribution function encoding the knowledge of the prior and the information encoded in the likelihood of the data Pr(p|D,I) ∝ Pr(p|I)L(p, D).

Standard techniques to achieve this result are fully analytical (e.g., for some linear regression), approximated (e.g., asymptotic approximation, Laplacian approximation, Gaussian approximation, etc.), iterative (e.g., Levenberg-Marquardt), or fully numerical (e.g., simulated annealing genetic algorithms). The choice between these techniques depends on the nature of the problem. Here, Laplacian approximation with hyperparameters are used (Hutter et al. 2011; Murphy 2012; Theodoridis 2015), as a few of the mathematical models that are considered herein are nested, to solve the inference problem (i.e., to search for the optimal set of parameters p that best represent the data). In order to confirm the inference results and to perform the Bayesian model comparison numerically, the results are both tested against the nested-sampling approach to the global likelihood (hereafter evidence) (Skilling 2004; Mukherjee et al. 2006; Feroz and Hobson 2008) and the Differential Evolution (Feoktistov 2006; Goode and Annin 2015) with up to aggressive scaling factors (≤ 0.9) and cross probabilities (≥ 0.1). For the Bayesian model comparison, the nested-sampling-based will embed the results in a natural framework.

Finally, a substantial limitation is noted in the fitting procedure from the sparse and irregular temporal sampling in the clinical data. This irregularity impacts the parameter space exploration due to the lack of condition on the PSA trend’s derivative. The partial derivative ∂ₜc_{PSA}(p;t) is not smooth, thus inhibiting using some straightforward optimization techniques based on the PSA curves’ gradients or convexity (Theodoridis 2015).

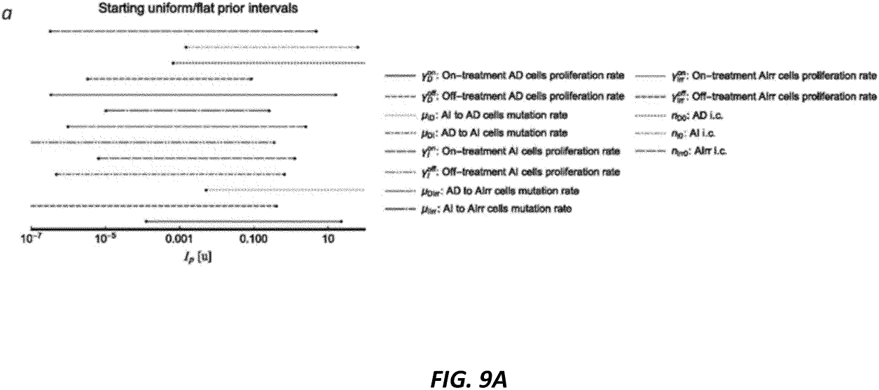

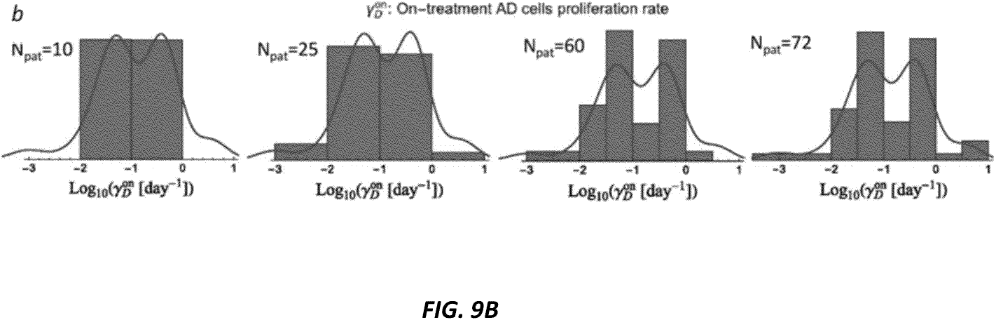

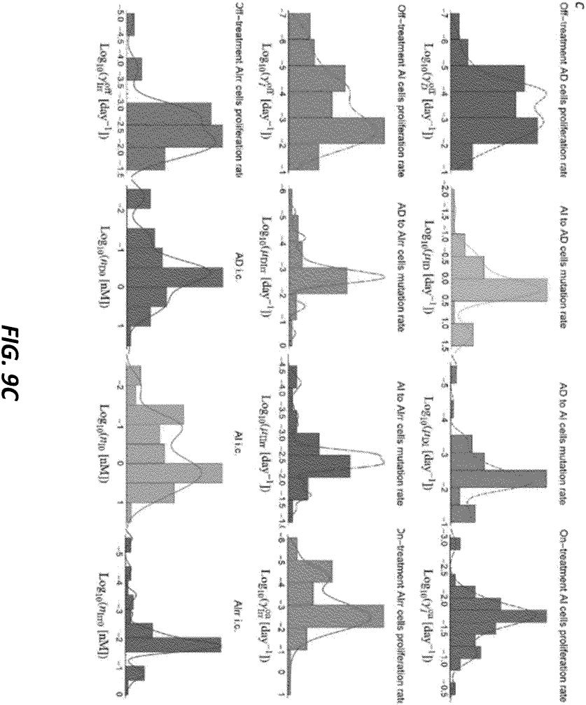

Referring now to FIGS. 9A-C, a schematic diagram depicting model prior development in provided. The depicted examples refer to the model by Hirata, Bruchovsky, and Aihara 2010 and its 13 defining parameters. A similar technique is adopted for the other models. FIG. 9A depicts an initial bounded flat prior. FIG. 9B depicts evolution of prior development for

$${γ}_{D}^{o n} \left[d a {y}^{- 1}\right)$$

as the number of patients analyzed is increased (Npat= {10,25,60,72} respectively). FIG. 9C depicts final priors for the remaining 12 parameters (colors correspond to those shown in FIG. 9A).

While robust approximations or numerical tools have been adopted for the Bayesian framework, special attention is paid to the use of priors. As mentioned, Bayesian inference requires the use of the priors, Pr(p|I), for parameter estimation. With initially unknown priors, uniform priors are implemented over the parameters’ full ranges (FIG. 9A). By requiring all model parameters to be positive, it can be assumed that the Heaviside step function θ = θ(p) as (unnormalized) prior, this approach is generally referred to as “improper prior” as it is unbounded above, it cannot be normalized, and therefore does not have a mean, standard deviation, median, or quantiles. An upper bound for each parameter is set to be p < pₘₐₓ with a max value pₘₐₓ < +∞ ∀p strictly. An alternative functional tested is the non-informative Jeffreys prior,

$$\text{Pr} \left(\left(\text{p}\right| I\right) ∝ \sqrt{\text{det} \left(\text{F} \left(\text{p}\right)\right)}$$

with F symbol referring to the Fisher Information matrix (Jeffreys 1946) and “det” to the matrix determinant.

Extra than testing with flat/Jeffreys priors, in the numerical nested sampling approach, the parameter space is explored logarithmically to avoid divergences, and once a statistically significative sample is reached, i.e., above the Poisson-noise fluctuation

$$\left(∼ \sqrt{{N}_{\text{pat}}}\right)$$

shaping the posterior PDF, the posterior is implemented as a prior for the patients analyzed in the dataset; finally, by implementing a recursive determination of the prior, as depicted in FIGS. 9B and 9C. Further details can be found in Pasetto et al., 2021, where Bayesian analysis of retrospective data to guide clinical decisions is discussed.

Only IAD models are considered due to current clinical interest. Each model is presented and justified in a biological and mathematical sense in the original papers where the models were first presented, and the reader is referred to them for detailed model derivations. Similarly, the sensitivity analysis of the model parameters is presented in each paper individually, and are elaborated on herein only where necessary. The relapsing patient set is referred to as Ω-set and not relapsing to as relapse ¬Ω-set.

The individual IAD data is parameterized with a patient-specific control function Tₚₛ defined as follows: Tₚₛ(t) =

$${T}_{p s} \left(t\right) = {\sum }_{i = 1}^{n} {1}_{{τ}_{i}} \left(t\right) , 0 < t ∈ \left[{t}_{min} , {t}_{max}\right]$$

with tₘᵢₙ and tₘₐₓ minimum and maximum patient-specific treatment under consideration (e.g., FIG. 8A) and tₘᵢₙ generally after the first treatment drop; n ≥ 1 is the number of intervals τᵢ considered. τⱼ ⊆ ]tₘᵢₙ,tₘₐₓ[∀i is referred to as the “i^{th} treatment cycle,” and 1_{τi} is the indicator function () for the interval τᵢ (defined as 1_{τi} = 1 for t ∈ τᵢ, 0 otherwise). Note that in this case the indicator function 1_{τi} is not-continuous scalar function, but traditionally indicated with bold characters even if it is not a matrix or a vector. Compact set notation is used here, e.g., 0 < t ∈ [tₘᵢₙ,tₘₐₓ] means all the possible values of t, positive, between tₘᵢₙand tₘₐₓ, i.e., 0 < tₘᵢₙ ≤ t ≤ tₘₐₓ. Open brackets will exclude the borders, e.g., soon after τᵢ ⊆ ]tₘᵢₙ,tₘₐₓ[ means that the interval τᵢ, e.g., τ = [a,b] is properly included between tₘᵢₙ and tₘₐₓ, but the limits of tₘᵢₙand tₘₐₓ are excluded: tₘᵢₙ < τᵢ < tₘₐₓ. This allows us to work with the domain of existence of the indicator functions but arbitrarily truncate it at tₘᵢₙor tₘₐₓ.

For modeling purposes, the weights/errors, eᵢ for each data i, have been assigned either uniformly eᵢ = cnst ∀i or with a linear decreasing relevance from the last PSA concentration c_{PSA} peak, say ĉ_{PSA} (e.g., in τ₃ of FIG. 8A) with

$${e}_{i} = {\left|{c}_{P S {A}_{i}} - {\hat{c}}_{P S A}\right|}_{t = \hat{t}}$$

at t = t and eᵢ =

$$\left|{\hat{c}}_{P S A}\right| - \left|\hat{t} - t\right| + \left|{c}_{P S {A}_{i}}\right|$$

for t ≠ t Finally, sensitivity analysis is performed on all the models included here.

### Ideta Et Al. 2008

The model by Jackson (Jackson 2004) can be considered the continuous ADT model prototype. Its extension to IAD therapy of interest was presented by Ideta et al. (Ideta et al. 2008). In this model (hereafter, I08), the authors drop the dependence of Jackson’s model on the spatial distribution, which is of theoretical interest not resolved in clinical PSA data. Model simulations predict that intermittent ADT can only prevent progression if normal androgen levels decrease the growth rate of AI cells, which may be biologically unlikely since AI cells have androgen receptors with increased sensitivity (Grossmann et al. 2001). Consider the I08 model in the following form:

$$\begin{matrix}\frac{d {n}_{D}}{d t} = \left({γ}_{D} - {δ}_{D} - {μ}_{D I}\right) {n}_{D} , \\ \frac{d {n}_{I}}{d t} = {μ}_{D I} {n}_{D} + \left({γ}_{I} - {δ}_{I}\right) {n}_{I} ,\end{matrix}$$

with initial conditions n_{D}(t_{0D}) = n_{D0}, n_{I}(t_{0I}) = n_{I0}. Note how, in general t_{0D} ≠ t_{0I}. As previously mentioned, n_{D} and n_{I} are the androgen-dependent and -independent population number of cells (or concentration). γᵢ and δᵢ,i ∈ {D,I} are growth and apoptosis rates for AD and AI cells, given respectively by:

$$\begin{matrix}{γ}_{D} = {γ}_{D max} \left({γ}_{D A} + \left(1 - {γ}_{D A}\right) \frac{{c}_{A}}{{c}_{A} + {k}_{D A γ}}\right) , {γ}_{I} = 1 - \left(1 - \frac{{δ}_{I A}}{{γ}_{I A}}\right) \frac{{c}_{A}}{{c}_{A 0}} , \\ {δ}_{D} = {δ}_{D max} \left({δ}_{D A} + \left(1 - {δ}_{D A}\right) \frac{{c}_{A}}{{c}_{A} + {k}_{D A δ}}\right) , {δ}_{I} = 1.\end{matrix}$$

In Eq. (19) γ_{Dmax} and δ_{Dmax} are the maximal AD proliferation and apoptosis rates, δ_{DA} is a control parameter on the effect of low androgen levels on the AD apoptosis rate, k_{DAγ} ≠ 0 is the AD half-saturation rate, k_{DAδ} ≠ 0 is the AD apoptosis rate dependence on androgen. Finally, δ_{IA} and γ_{IA} ≠ 0 modulate hormonally patient failing death and growth. Mutation from AD to AI cells are allowed at a mutation rate:

$${μ}_{D I} = {μ}_{D I \text{max}} \left(1 - \frac{{c}_{A}}{{c}_{A 0}}\right) ,$$

thus, the mutation rate decreases as the androgen (here normalized at its homeostatic level c_{A0} ≠ 0) approaches its max value µ_{DImax}. A decoupled ODE model of the serum androgen concentration under treatment c_{A} is given by:

$$\frac{d {c}_{A}}{d t} = {δ}_{c A} \left({c}_{A 0} - {c}_{A}\right) - {δ}_{c A} {c}_{A 0} {T}_{p s} ,$$

with initial condition c_{A}(t_{0A}) = c_{A0} ≠ 0, where δ_{cA} is the androgen clearance rate. Here Tₚₛ = Tₚₛ(t) is the patient treatment-specific function as defined above. Finally, the PSA density concentration of interest to us, c_{PSA}, is a linear combination with weight wᵢ of the population densities

$${c}_{P S A} = {\sum }_{i ∈ \left\{D , I\right\}} {w}_{i} {n}_{i} .$$

Based on the original analysis of Ideta et al. and the available dataset, two versions of this model are explored. Namely, where δ_{IA} = γ_{IA}, i.e., γ_{I} = cnst (hereafter model I08A) and the original form of the equations (δ_{IA} ≠ γ_{IA}) (hereafter model I08B).

### I08A in the Context of the Data

It is noted that the system of equations (hereafter SoE) composed by Eqs. (18), (19), (20), and (21), decouples in the androgen concentration c_{A}. The analysis of the system results in a line of infinite equilibria on the intersection of the plane n_{D} = 0 with the plane c_{A} = c_{A0} — c_{A0}Tₚₛ in the space of phase-state variables (n_{D},n_{I},c_{A}). Thus, c_{A} = c_{A0} off-treatment and c_{A} = 0 on-treatment. Standard linear stability analysis (Wiggins 2003) shows that the Jacobian of the system produces a null generalized eigenvalue λ₁, = 0, a negative one λ₂ = —δ_{A}, and a more complicate third generalized eigenvalue that takes, off-treatment, the elegant form:

$${λ}_{3}^{\text{off}} = {γ}_{\text{Dmax}} + \frac{\left({γ}_{\text{DA}} - 1\right) {γ}_{\text{Dmax}} {k}_{\text{D} γ / 2}}{{c}_{\text{A0}} + {k}_{\text{D} γ / 2}} - {δ}_{\text{Dmax}} - \frac{\left({δ}_{D A} - 1\right) {δ}_{\text{DMAX}} {k}_{D δ / 2}}{{c}_{\text{A0}} + {k}_{D δ / 2}} .$$

The sign of

$${λ}_{3}^{\text{off}}$$

can be evaluated for the best-fit parameter values that result from the inference works in the patients’ cohort considered here, resulting in being always positive for all the patients. Therefore, the above-found equilibria lines represent a 1D nonstable manifold, and further investigations (e.g., in the context of the central manifold theory) are not of additional interest to us.

The characteristics of the present dataset in the context of this model may be further exploited by using the decoupled nature of the serum androgen concentration c_{A}. All the patients are considered from their first cycle of treatment, starting with Tₚₛ(t) = 1 for t ɛ τ₁. Hence, it can be emulated with a Heaviside step function Tₚₛ = θ(-t) a cycle of treatment followed by the off-treatment period for a suitable cyclic interval (on-off, on-off, on-off, and so forth) around the off-treatment start, set at t = 0. Within this approach, the general solution of is algebraic and reads:

$${C}_{A} \left(t\right) = {c}_{\text{A0}} {e}^{- {δ}_{Z} \left(t + 1\right)} \left({e}^{{δ}_{A}} θ \left({e}^{{δ}_{A} t} - 1\right) + 1\right) .$$

This equation is monotonic on the two phases on/off-treatment because the derivative dc_{A}/dt = c_{A0}δ_{Ae}^{-δA(t+1)}(e^{δA}θ - 1) — 1) is never null neither for t < 0, i.e., on-treatment nor for t ≥ 0, off-treatment. By splitting the treatment in on/off-time, the bilinear map c_{A} = c_{A}(t) in t = t(c_{A}) can be reversed. For example, in the instant case, it reads

$$t = - \frac{1}{{δ}_{A}} log \left(\frac{{c}_{A}}{{c}_{A 0}}\right) + {δ}_{A}$$

on-treatment and

$$t = \frac{1}{{δ}_{A}} log \frac{{c}_{A 0} \left({\text{e}}^{- {δ}_{A}} - 1\right)}{{c}_{A} - {c}_{A 0}}$$

off-treatment for c_{A} ≠c_{A0}, and c_{A} ≠0 and δ_{A} ≠0. The resulting SoE could be considered as function of the variable c_{A} to reach a fully algebraic solution of the system by taking the ration of

$$\frac{d {n}_{D}}{d {c}_{A}} / \frac{d {n}_{I}}{d {c}_{A}}$$

. Nevertheless, it is more fruitful to look at the trend of Eq. (23) as obtained by the

$$\frac{d {n}_{D}}{d {c}_{A}} / \frac{d {n}_{I}}{d {c}_{A}}$$

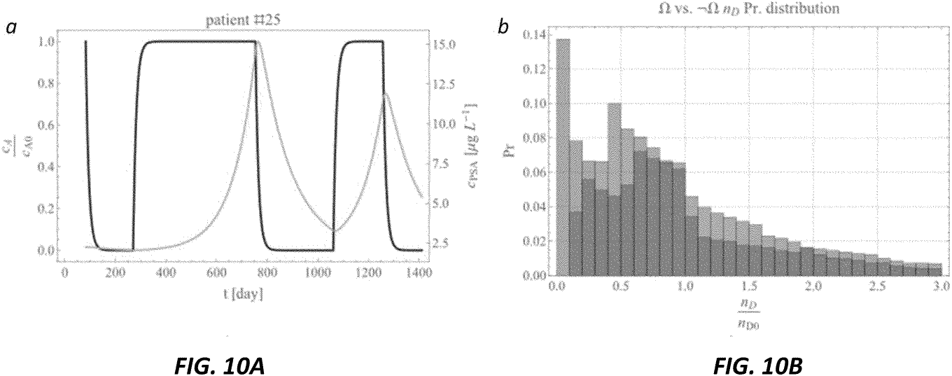

best fit procedure introduced in the next section. Eq. (23) is exploited to obtain the probability distribution function (PDF) of the orbits over all the sets of patients remapping each cycle over the phase-space section (O, n_{D}, n_{I}). Advantage is taken by the sharp c_{A} passage from its homeostasis value c_{A0} to null and vice versa in conjunction with the bijection map just found. FIG. 10A shows the c_{A} profile for a representative patient. While time is a monotonic increasing function, the map considered is one-to-one only over the treatment cycle Tₚₛ = 1 and the off-cycle Tₚₛ = 0 respectively, and in these two tracks the SoE can be written as n_{D} = n_{D}(t(c_{A})) = n_{D}(c_{A}) and n₁, = n₁(c_{A}).As c_{A}sharply switches from c_{A} = c_{A0} and c_{A} = 0, the present disclosure is limited to a first order solution of the SoE. After simple algebra, the approximate solution of the SoE in the form:

$$\begin{matrix}{n}_{D} ≃ {n}_{\text{D} 0} - \\ \frac{1}{{c}_{\text{A} 0} {δ}_{A}} {n}_{\text{D} 0} \left({c}_{A} - {c}_{\text{A} 0}\right) \left(\frac{{γ}_{\text{Dmax}} \left({c}_{\text{A} 0} + {γ}_{A} {k}_{\text{D} γ / 2}\right)}{{c}_{\text{A0}} {k}_{\text{D} γ / 2}} - \frac{{δ}_{\text{Dmax}} \left({c}_{\text{A0}} + {δ}_{D} {k}_{\text{D} δ / 2}\right)}{{c}_{\text{A0}} + {k}_{\text{D} δ / 2}}\right) , \\ {n}_{I} ≃ {n}_{\text{I0}} ,\end{matrix}$$

to the first order in c_{A} (and where ≃means asymptotic-to). As evident, the second equation remains close to its initial value n_{I0} , while the first is perturbed away, suggesting that the PDF of the dataset can be sampled for fixed values in n₁ around n_{I0}, and then investigate the PDF as sampled from the best fit obtained by the patient in the trial with Eq. (24). The results are shown in FIG. 10B. The trend of the two distributions for the development of resistance and continuing response patients is comparable as above the starting value n_{D} = n_{D0}, while the trend diverges for smaller values n_{D} . Because it can be assumed n_{D} is a proxy for c_{PSA} at small values of nᵢ, as evicted from Eq. (22) and (23), if the model correctly interprets the data, then a patient with an initial PSA-drop below 10% of its initial value is highly likely to be a continuous responder. The risk of resistance development grows to about 50% when the initial drop in PSA is around 30%.

### I08B In The Context of The Data

For I08B, where δ_{IA}≠γ_{IA}, the ratio presented in Eq. (20) evidences the structural non-identifiability of the SoE. The treatment of the equilibria and their stability is more straightforward in this model form than in I08A. The only equilibrium point is given by {n_{D}, n_{I}, c_{A}}_{eq} = {0,0, c_{A0} - c_{A0}Tₚₛ} with generalized eigenvalues λᵢof the Jacobian at the equilibrium given by λ₁ =

$$\left({T}_{p s} - 1\right) \left({γ}_{\text{IA}} - {δ}_{\text{IA}}\right) {γ}_{I A}^{- 1} \text{with} {γ}_{I A} \neq 0 , {λ}_{2} = {λ}_{2}^{\text{I08A}} \text{and} {λ}_{3} = {λ}_{3}^{\text{I08A}}$$

.Following the I08A assumptions, the model is investigated under the conditions (δ_{Dmax} > γ_{Dmax}, (δ_{DA} > 1, and by requiring that µₘₐₓ < γ_{I} -δ_{I} to avoid the annihilation of the populations. Under these conditions, it can proven that λ₁ ≤ 0 and λ₂ ≤ 0, and that for λ₃ it holds the same consideration as for I08A due to the non-stable nature of the resulting equilibrium manifold.

Analogous consideration on the non/relapsing treatment holds for I08B as for I08A, but with more straightforward treatment for I08B than for I08A: the two equilibria at homeostasis c_{A} = c_{A0} and at null androgen concentration, c_{A} = 0, attract the dynamics as for I08A and self-explain the orbit profiles. Therefore, identical results from the inference of I08B on patients’ trials can be obtained for the PDF but are not depicted again.

### Eikenberry Et Al. 2010

The model developed by Eikenberry et al. (Eikenberry et al. 2010, hereafter E10) was an attempt to describe the interaction between testosterone (T, the primary androgen in the serum), its enzyme 5a-reductase to dihydrotestosterone (DHT), and their binding (T:AR and DHT:AR) with the androgen receptors (AR) in the prostate. Because of model E10’s versatility, it is included in the IAD treatment model comparison. Of note, the authors have not proposed the model to fit data, and here E10 is reinterpreted beyond the scope of the original paper. The modulation due to intermittent IAD is assumed in testosterone time modulation. While a linear relation might not be readily available from the literature between testosterone and PSA level (Elzanaty et al. 2017), the testosterone concentration n_{T}, is recoded in E10 as follows:

$$\frac{d {n}_{T}}{d t} = {n}_{T} \left({δ}_{T} - \frac{{μ}_{\text{cat}} {n}_{5 α}}{{k}_{M} + {n}_{T}} - {κ}_{T : R} {n}_{R}\right) + {δ}_{T : R} {q}_{T : R} - \left({T}_{\text{ps}} - 1\right) ϒ \left({n}_{\text{S}}\right) ,$$

which is coupled with the original system of equations:

$$\begin{matrix}\frac{d {n}_{R}}{d t} = {n}_{R} \left({γ}_{R} - {δ}_{R} - {κ}_{D H T} {n}_{D H T} - {κ}_{T : R} {n}_{T}\right) + {δ}_{D H T : R} {q}_{D H T : R} + {δ}_{T : R} {q}_{T : R} , \\ \frac{d {n}_{D H T}}{d t} = \frac{{μ}_{c a t} {n}_{5 α} {n}_{T}}{{k}_{M} + {n}_{T}} - {n}_{D H T} \left({δ}_{D H T} + {κ}_{D H T} {n}_{R}\right) + {δ}_{D H T : R} {q}_{D H T : R} , \\ \frac{d {q}_{T : R}}{d t} = {κ}_{T : R} {n}_{R} {n}_{T} - {δ}_{T : R} {q}_{T : R} , \\ \frac{d {q}_{D H T : R}}{d t} = {κ}_{D H T} {n}_{D H T} {n}_{R} - {δ}_{D H T : R} {q}_{D H T : R} ,\end{matrix}$$

with five nominals initial conditions: n_{R0} = n_{R}(t_{0R}), n_{T0} = n_{T}(t_{0T}), n_{DHT0} = ⁿDHT(^{t}0DHT), ^{q}_{T:R0} = ^{q}_{T:R}(^{t}_{0T:R}) and q_{DHT:R0} = q_{DHT:R}(t_{0DHT:R}). Here, the treatment function Tₚₛ modulates testosterone influx into the prostate-function γ(nₛ) original in E10 and that is adopted here, where nₛ is the testosterone serum concentration. Furthermore, the androgen receptor concentration n_{R} and the dihydrotestosterone concentration n_{DHT} are considered together with two quota concentrations q_{T:R} and Q_{DHT:R} (Droop 1968), here, taken to be the T:AR complex and the DHT:AR complex concentration, respectively. γ_{R} is the AR production rate, δ_{R} is the AR degradation rate, δ_{T} the testosterone-specific degradation rate, and δ_{DHT} the dihydrotestosterone degradation rate. The mass-action constants for the androgen-dependent component (testosterone) and dihydrotestosterone binding the AR are

$$\left\{{κ}_{a}^{T} , {κ}_{d}^{T} , {κ}_{a}^{\text{DHT}} , {κ}_{d}^{\text{DHT}}\right\}$$

, and the 5α reductase converts T to DHT by Michaelis-Menten enzyme kinetics with concentration n₅ₐ, turnover number µ_{cat} and constant k_{M} ≠ 0.

### The Model In The Context Of The Data

If a ≡ µ_{cat}n₅ₐ - δ_{T}k_{M}, b ≡ (1 - Tₚₛ)γ(nₛ), and

$$\text{Δ} ≡ \sqrt{{\left(a + b\right)}^{2} - 4 b {δ}_{T} {k}_{M}} ,$$

then two critical points can be isolated at the intersection of the nullclines hyperplanes of the phase-

$$\begin{matrix}{\left\{{n}_{R} , {n}_{T} , {n}_{\text{DHT}} , {q}_{T : R} , {q}_{\text{DHT:} R}\right\}}_{\text{eq}}^{\left(1 , 2\right)} = \\ \left\{0 , 0 , 0 , \frac{- a - b ∓ \text{Δ}}{2 {δ}_{T}} , \frac{- a + b \pm \text{Δ}}{2 {δ}_{\text{DHT}}}\right\}\end{matrix}$$

holds as space. On-treatment, the first point

$$\begin{matrix}{\left\{{n}_{R} , {n}_{T} , {n}_{\text{DHT}} , {q}_{T : R} , {q}_{\text{DHT:} R}\right\}}_{\text{eq}}^{\left(1 , 2\right)} = \\ \left\{0 , 0 , 0 , \frac{- a - b ∓ \text{Δ}}{2 {δ}_{T}} , \frac{- a + b \pm \text{Δ}}{2 {δ}_{\text{DHT}}}\right\}\end{matrix}$$

soon as ∓a + b + Δ≤ 0 ∧ ∓a + Δ ≤ b. While only the second of these equilibria is of biological interest, it is not a stable equilibrium. Obtaining the complete set of generalized eigenvalues requires a cumbersome solution of three cubic equations, yet the check for the stability requires much less effort once it is realized that one of the generalized eigenvalues from the characteristic equations reads simply

$${δ}_{T} - \frac{4 {δ}_{T} {}^{2} {μ}_{\text{cat}} {k}_{M} {n}_{5 α}}{{\left(a + b - \text{Δ} - 2 {δ}_{R} {k}_{M}\right)}^{2}}$$

where a + b — Δ — 2δ_{T}k_{M} ≠ 0 and that it proves to be always positive for all the inference results in the trial patients.

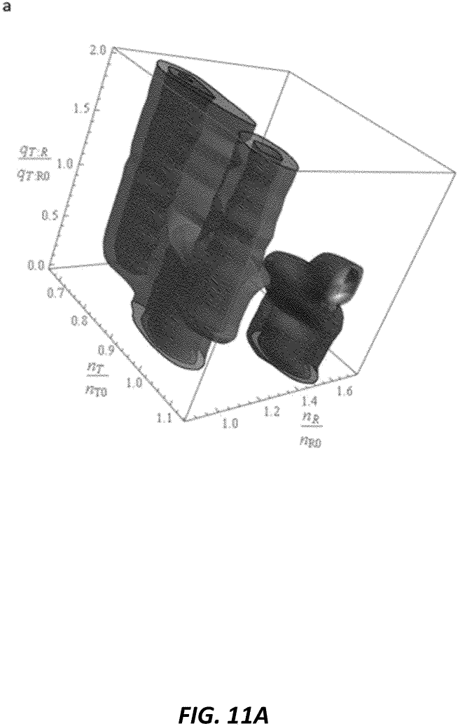

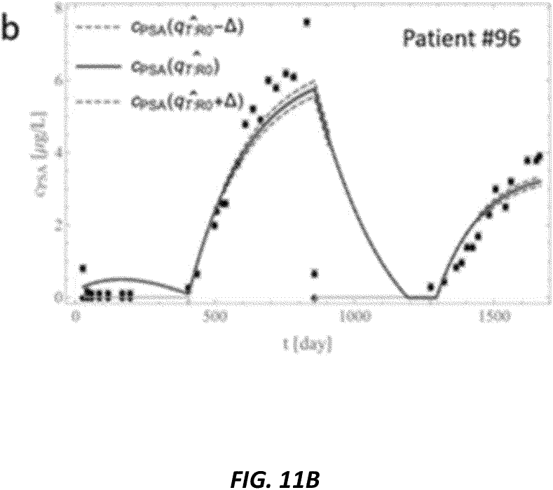

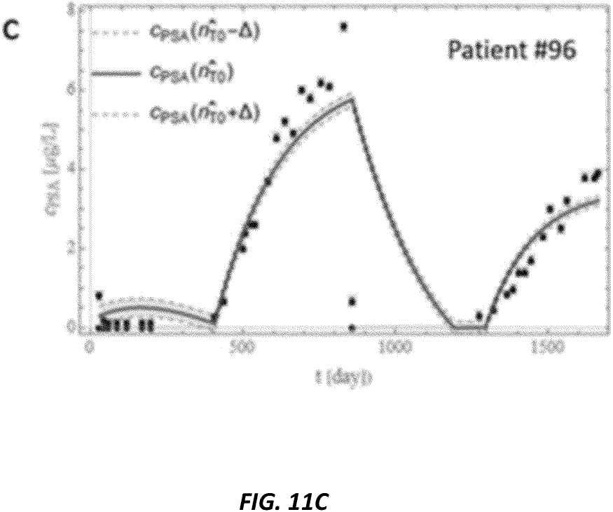

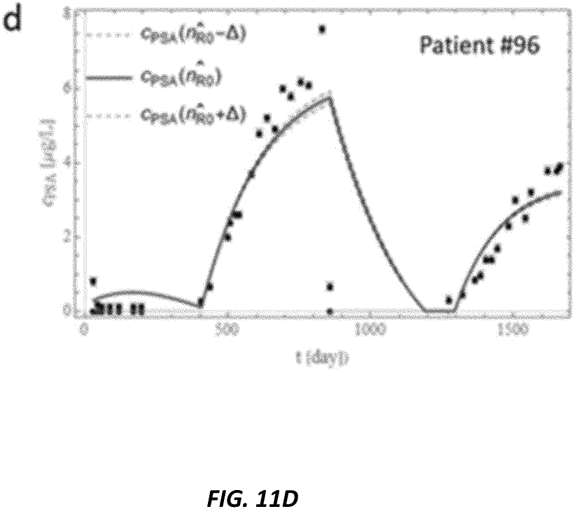

Finally, the model could represent an essential instrument for investigating the relapsing mechanism evidenced in some patients, which remains one of the goals of this work for its potential clinical implications. Three over five state variables are identified by inspecting the phase-state space with a striking separation between Ω and ¬Ω. FIG. 11A shows the 3D probability distribution function of n_{T}, n_{R}, and q_{T:R}. The density map of the temporal evolution of Ω and ¬Ω sets clusters (over the orbital evolution spanned by the patients analyzed) on a well distinct area of the phase-space, splitting in the n_{T} vs. n_{R} space and at least partially in the orthogonal q_{T:R} space.

In FIGS. 11B-D, the DDM is exploited for sensitivity analysis to track the time dependence of the sensitivity

$${S}_{i j} ≡ \frac{\partial {c}_{P S A} \left(t , \hat{p}\right)}{\partial {p}_{j}}$$

computed at the best fit parameter values p̂, where pⱼ = {n_{T0}, n_{R0}, q_{T:R0}} respectively. As shown in FIGS. 11B-D, a slight variation of the parameters does not dramatically affect the trend of c_{PSA}. Thus, there is minimal sensitivity of c_{PSA} to the parameters. This result shows that the PDF of the combination of parameters investigated might be an excellent tool to explore the origin of the resistance with the E10 model.

The sensitivities were computed using DDM, which was mentioned herein and is reported more in Supplement A. As evident from FIGS. 11B-D, different parameters have different sensitivity on a different phase orbit with n_{T0} more sensitive under treatment and n_{R} or q_{T:R} more sensitive out of treatment. DDM not only demonstrates the stability of the results obtained but also adds extra information on when a model is sensitive to a parameter change. This result is significant when dealing with models with varying behavior on and off-treatment.

### Hirata, Bruchovsky, and Aihara 2010

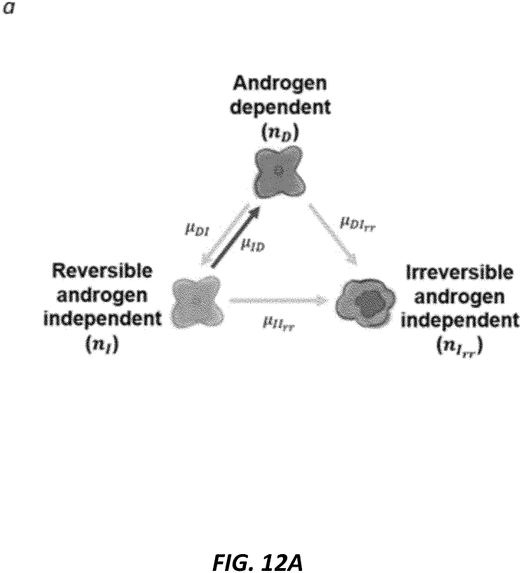

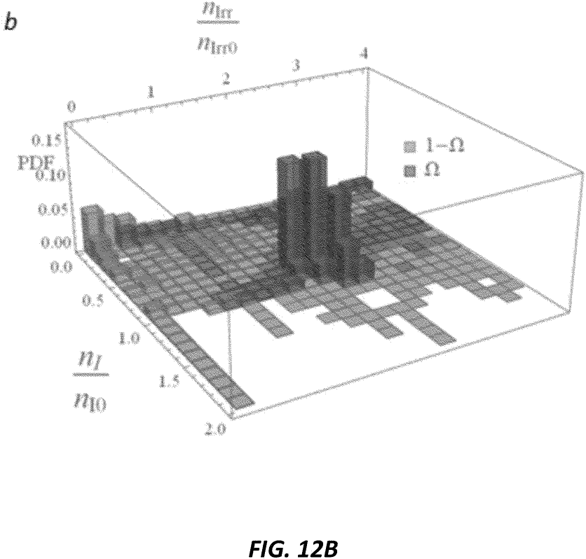

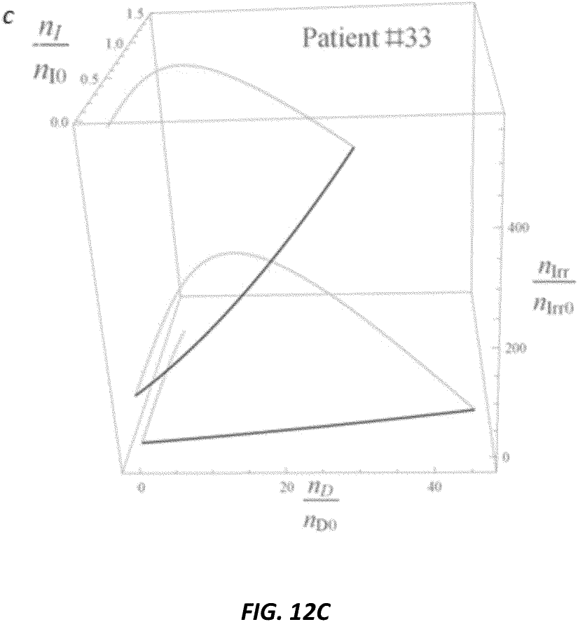

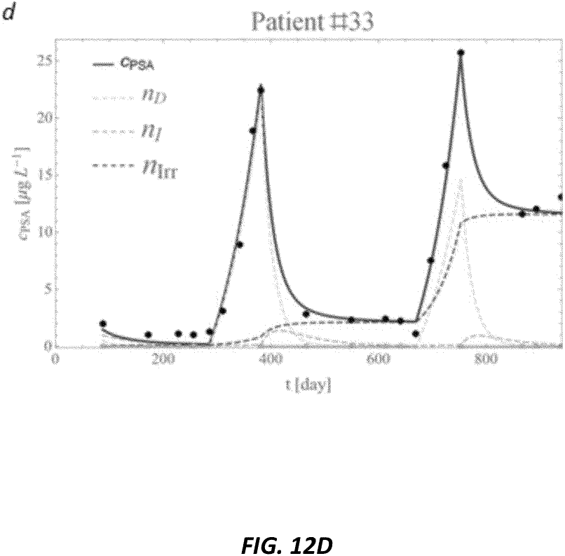

A series of studies (Tanaka et al. 2010; Hirata et al. 2012; Hirata and Aihara 2015) motivated the model by Hirata et al. 2010 (hereafter model H10) to capture intermittent ADT dynamics. The model is based on the coupled AD-Al population cells, supplemented with a population of irreversible Al cells, AI-Irr representing the first 3-compartments model in the literature (FIG. 12A). Here the mathematical formulation is reported in the proposed framework’s formalism and refer to the original paper for a detailed model description. The SoE reads with the generalized notation reads:

$$\begin{matrix}\frac{d {n}_{D}}{d t} = n {}_{D} \left({T}_{p s} \left({γ}_{D}^{\text{on}} - {γ}_{D}^{\text{off}}\right) + {γ}_{D}^{\text{off}}\right) + {μ}_{I D} \left(1 - {T}_{p s}\right) {n}_{I} , \\ \frac{d {n}_{I}}{d t} = {μ}_{D I} {T}_{p s} {n}_{D} + {n}_{I} \left({T}_{p s} \left({γ}_{I}^{\text{on}} - {γ}_{I}^{\text{off}}\right) + {γ}_{I}^{\text{off}}\right) , \\ \frac{d {n}_{I r r}}{d t} = {μ}_{D I r r} {T}_{p s} {n}_{D} + {μ}_{I I r r} {T}_{p s} {n}_{I} + {n}_{I r r} \left({T}_{p s} \left({γ}_{I r r}^{\text{on}} - {γ}_{I r r}^{\text{off}}\right) + {γ}_{I r r}^{\text{off}}\right)\end{matrix}$$

n_{D}(t_{oD}) = n_{D0}, n_{I}(t_{0I}) = n_{I0}, n_{Irr}(t_{0Irr}) = n_{Irr0}, where terms retain the identical biological meaning as previously described and the two irreversible and reversible changes in the Al cell population are considered with the relative growth rate γᵢ^{on/off} on and off-treatment with i ɛ {D, I, Irr}. The serum concentration is computed as in Eq. (22) for i ɛ {D, I, Irr}.

### The Model in the Context of the Data

During both on and off treatment cycles, nullclines analysis leads to {n_{D}, n_{I}, n_{Irr}}_{eq} = {0,0,0} as the only equilibrium point. By setting

$$a ≡ {γ}_{D}^{\text{off}} + {γ}_{I}^{\text{off}} b ≡ {γ}_{D}^{o n} + {γ}_{I}^{\text{on}} , c ≡$$

$${γ}_{D}^{\text{off}} - {γ}_{I}^{\text{off}} \text{and} d ≡ {γ}_{D}^{\text{on}} - {γ}_{I}^{\text{on}} ,$$

with the discriminant Δ implicitly defined by the relation Δ² = c² + Tₚₛ(Tₚₛ((c — d)² — 4µ_{DI}µ_{ID}) — 2c(c - d) + 4µ_{DI}µ_{ID}), the generalized eigenvalues of the Jacobian at the equilibrium as

$${λ}_{1} = {γ}_{I r r}^{\text{off}} + {T}_{p s} \left({γ}_{I r r}^{\text{on}} - {γ}_{I r r}^{\text{off}}\right)$$

and the other two λ_{2,3}can be written in a compact form as

$${λ}_{2 , 3} = \frac{1}{2} \left({T}_{p s} \left(b - a\right) + a \pm Δ\right) .$$

This result implies that the equilibrium is stable on-treatment and unstable off-treatment.

The phase space shows that responsive and resistant patients cluster differently on the phase-state variables. FIG. 12B shows that the probability density function for the best-fit patient groups around the initial value for n_{I} ≅ n_{I0} and n_{Irr} ≅ 2.1n_{Irr0}. Thus, the irreversible component of the model offers a potential tool to disentangle patient responses from the model fitting. As the resistant patients are expected to increase their irreversible cell component (i.e., asymptotically n_{Irr} ≻ n_{Irr0} with “≻” meaning asymptotic greater), it is noted that n_{I} « n_{I0} in responsive patients.

The model structure allows for the simulation of various PSA profiles thanks to the introduction of a new degree of freedom carried with the third compartment equations. FIG. 12C shows the phase-space plane for an example taken from the Ω set of patients (Patient #33), while shows the quality of the captured PSA concentration c_{PSA} profile achieved by this model.

### Portz, Kuang and Nagy 2012

The Portz et al. 2012 model is based on the cell quota concept (Droop 1968), which is modeled as:

$$\frac{d {q}_{i}}{d t} = \frac{{γ}_{max} \left({q}_{max} - {q}_{i}\right) \left(1 - {T}_{p s}\right)}{\left({q}_{max} - {q}_{i min}\right) \left({k}_{q / 2} - {T}_{p s} + 1\right)} - {δ}_{q} {q}_{i} + {γ}_{max} \left({q}_{i min} - {q}_{D}\right) ,$$

with q(t₀ᵢ) = q₀ᵢ for i ɛ {D, I}. The cell quota can grow to the maximum cell quota rate γₘₐₓ and degrades at a constant rate δ_{q}, with qₘₐₓ representing the shared max cell quota, νₘₐₓ the maximum cell quota uptake rate, qᵢₘᵢₙ < qₘₐₓ the minimum cell quota for androgen, and 1 ≠ k_{q/2} > 0 the uptake rate half-saturation level (Packer et al. 2011). The authors allow mutation between both cell populations, from AD to Al and vice versa, at rates µ_{DI} and µ_{ID} given respectively by the Hill’s equations of an index m = 2:

$${μ}_{D I} \left(q\right) = {μ}_{D I \text{max}} \frac{{k}_{D I / 2}^{m}}{{q}^{m} + {k}_{D I / 2}^{m}} , {μ}_{D I} \left(q\right) = {μ}_{I D \text{max}} \frac{{q}^{m}}{{q}^{m} + {k}_{I D / 2}^{m}} ,$$

where µ_{DImax} is the maximum AD to Al mutation rate, µ_{IDmax} is the maximum Al to AD mutation rate, and

$${k}_{D I / 2}^{m} \text{and} {k}_{I D / 2}^{m}$$

are the cells mutation rate half-saturation level. The model follows the evolution of AD/AI cell populations, n_{D} and n_{I} respectively, with the following equations:

$$\begin{matrix}\begin{matrix}\frac{d {n}_{D}}{d t} = {n}_{D} \left(- {δ}_{D} - {μ}_{D I max} \frac{{k}_{D I / 2} {}^{2}}{{k}_{D I / 2} {}^{2} + {q}_{d} {}^{2}} + {γ}_{max} \left(1 - \frac{{q}_{D min}}{{q}_{D}}\right)\right) + \\ {μ}_{I D max} {n}_{I} \frac{{q}_{1}^{2}}{{k}_{I D / 2} {}^{2} + {q}_{1}^{2}} ,\end{matrix} \\ \begin{matrix}\frac{d {n}_{I}}{d t} = {n}_{1} \left(- {δ}_{1} - {μ}_{I D max} \frac{{q}_{i} {}^{2}}{{k}_{I D / 2} {}^{2} + {q}_{I} {}^{2}} + {γ}_{max} \left(1 - \frac{{q}_{I min}}{{q}_{I}}\right)\right) + \\ {μ}_{D I max} {n}_{D} \frac{{k}_{D I / 2} {}^{2}}{{k}_{D I / 2} {}^{2} + {q}_{D}^{2}} ,\end{matrix}\end{matrix}$$

for qᵢ(t) ≠ 0∀t and i.c. n_{D}(t_{0D}) = n_{D0} and n_{I}(t_{0I}) = n_{I0}. The cell apoptosis and proliferation rates are respectively given by δᵢ and γᵢ for i = {D, I}. The authors model the quota for both AD and Al cell populations independently. In general, it is assumed that q_{Imin} < q_{Dmin} to ensure that Al cells have a greater proliferation capacity in low androgen environments and n_{D}(t_{0D}) ≅ 0 with t_{0D} soon after treatment as well as n_{I}(t_{0I}) ≅ 0 at t_{0I} at the beginning of the first treatment. Furthermore, a communal maximum proliferation rate γₘₐₓ between the two populations is assumed. Both AD and Al cells produce PSA at a baseline rate γ_{PSA0} under the androgen dependence specified by:

$$\begin{matrix}\frac{d {c}_{P S A}}{d t} = {n}_{D} \left({γ}_{P S A 0} + \frac{{γ}_{P S A , D q {D}^{2}}}{{k}_{P S A , D / 2} {}^{2} + {q}_{D} {}^{2}}\right) - {C}_{P S A} {δ}_{P S A} + \\ {n}_{I} \left({γ}_{P S A 0} + \frac{{γ}_{P S A , I q {I}^{2}}}{{k}_{P S A , I / {2}^{2}} + {q}_{I} {}^{2}}\right) ,\end{matrix}$$

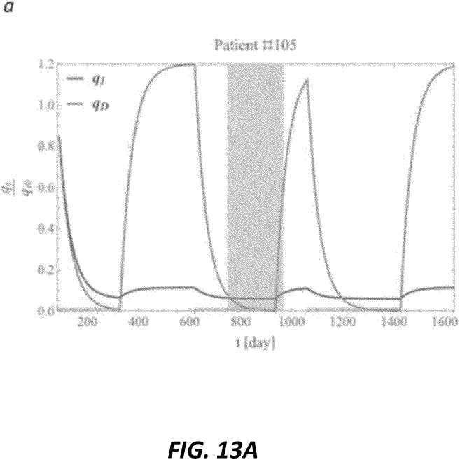

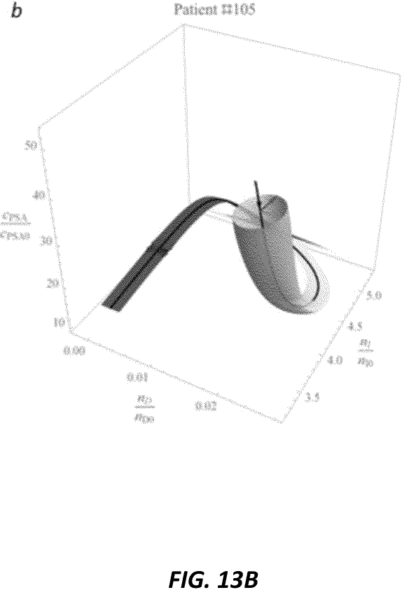

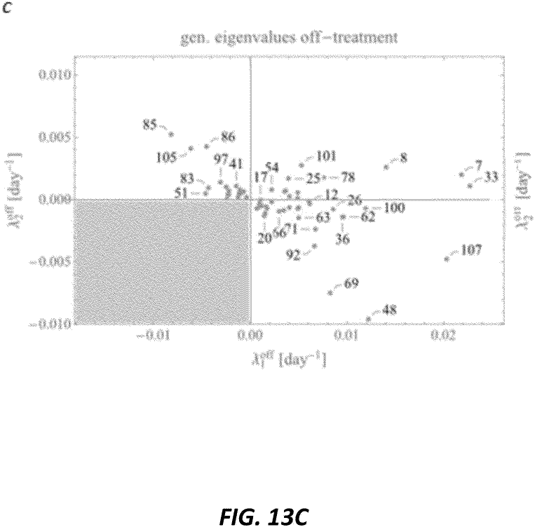

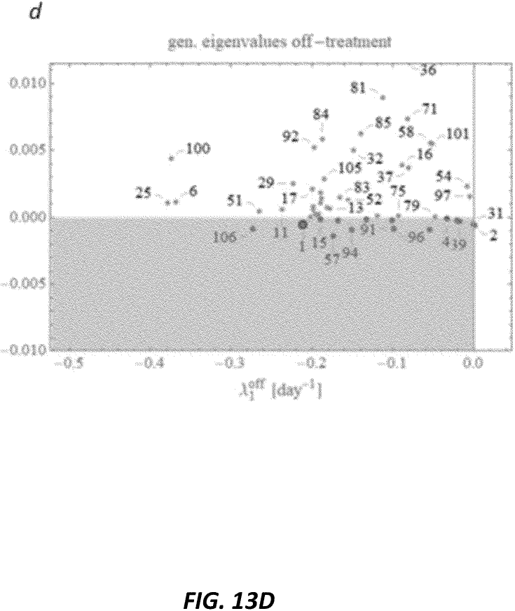

with c_{PSA}(t_{0PSA}) = c_{PSA0}, and where k_{PSA,i/2} are the half-saturation rates and γ_{PSA,i} the growth rates, for i = {D, I}. Several variants of this quota model can be found in the literature. In the present disclosure, only a couple of them are considered. A detailed comparison between (Hirata et al. 2010) and (Portz et al. 2012) can be found elsewhere (Everett et al., 2014). The model’s complexity is demonstrated with a tube plot (FIGS. 13A-B).

### P12A in the Context of the Data

The model is an extension of the models by Ideta et al., detailed above, where the equation of the quota decouples from the two cell populations behavior. Nevertheless, the quota evolution q = q(t), common to n_{D} and n_{I,} is generally smoother than c_{A} = c_{A}(t) in I08A or I08B hence not justifying the approximations worked out in those models. In P12A, the only equilibrium point is at

$${\left\{{n}_{D} , {n}_{I}\right\}}_{\text{eq}}^{\text{on/off}} = \left\{0 , 0\right\}$$

and for the decoupled quota equation at

$${q}_{\text{eq}}^{\text{on}} = \frac{{γ}_{\text{Dmax}} {q}_{min}}{{γ}_{\text{Dmax}} + {δ}_{q}} \text{and} {q}_{\text{eq}}^{\text{off}} =$$

$$\frac{{γ}_{D max} \left({k}_{q / 2} + 1\right) {q}_{min} \left({q}_{max} - {q}_{min}\right) + {q}_{max} {v}_{max}}{\left({k}_{q / 2} + 1\right) \left({γ}_{\text{Dmax}} + {δ}_{q}\right) \left({q}_{max} - {q}_{min}\right) + {v}_{max}}$$

for the on/off-treatments, respectively. The eigenvalues at this equilibrium point are real and negative along the direction of n_{D} and

$${n}_{I} : {λ}_{i} ∈ ℝ {}_{0}^{-}$$

, for i = 1,2 both on-and off-treatment. In the decoupled q direction, the generalized eigenvalues

$${λ}_{3}^{\text{on}} = - \left({γ}_{D max} + {δ}_{q}\right)$$

and

$${λ}_{3}^{\text{off}} = {λ}_{3}^{\text{on}} - \frac{{v}_{max}}{\left({k}_{q / 2} + 1\right) \left({q}_{max} - {q}_{min}\right)}$$

are always negative, leading to a node (attractor).

Nevertheless, it is noted that from the plot in FIG. 13C, how the best-fit solutions obtained from inference work for all patients with this model falls in the area where λᵢ > 0, for both i = 1 and i = 2, i.e., the presence of an attractor (off-treatment) is not noted. Therefore, patient dynamics never intercept an area of the parameters’ space defined by the hyperplane that would (eventually asymptotically) lead to the annihilation of the n_{D} and n_{I}, cell population, i.e., a steady-state or a reduction of the disease presence under the detection threshold. This plot is compared with the companion model in the next section, which simplifies P12A.

### P12B in the Context of the Data

In this model, the authors extend the use of the quota concept to both n_{D} and n_{I}, individually, i.e., fully exploiting Eq.(28), but retaining the same proliferation rate γₘₐₓ. The large number of parameters required by the model makes the posterior maximization time-consuming and computationally expensive in the Bayesian framework, especially in a fully numerical nested-sample approach (Skilling 2004) or Differential Evolution optimization (Feoktistov 2006; Goode and Annin 2015). For this reason, a first inference approach has been performed within Laplace approximation and followed up at the patient-specific level where judged necessary.

As in P12A, the P12B critical points are {nD, n_{I} c_{PSA}eq} = {0,0,0} both on- and off-treatment, while for the decoupled quota equation-stability points are found at

$${q}_{i , \text{eq}}^{o n} = \frac{{γ}_{max}}{{δ}_{q} + {γ}_{max}} {q}_{i min}$$

and

$${q}_{\text{i,eq}}^{\text{off}}$$

= a⁻¹µₘₐₓqᵢₘᵢₙ(k_{q/2} + 1)(qₘₐₓ — qᵢₘᵢₙ) + qₘₐₓνₘₐₓ with a ≡ νₘₐₓ — (k_{q/2} + 1)(δ_{q} + γₘₐₓ)(qᵢₘᵢₙ — qₘₐₓ) ≠ 0, δ_{q} ≠ 0 and γₘₐₓ ≠ 0 and for i ɛ I,D. As in P12A, the three generalized eigenvalues of the Jacobian at the equilibrium are always negative. Equations for the generalized eigenvalues

$${λ}_{i}^{\text{on/off}}$$

for i = 1,2 along the quota directions are analytically available but slightly cumbersome; vice versa, more interesting is the plot of

$${λ}_{i}^{\text{off}}$$

for i = 1,2 shown in FIG. 13D. The P12B solutions distribute a small number of patients in the P12A inaccessible area of double negative generalized eigenvalues (orange square in FIGS. 6c and 6d). In this zone of the P12B parameter space off-treatment, the model predicts a constrained (or asymptotically constrainable) tumor cell population. Finally, it is noted that P12A is nested in P12B. Thus, P12B always obtains a better score in the same data representation but suffers from overfitting. This problem is investigated further herein in the context of the Bayesian model comparison.

### Morken Et Al. 2014

In Morken et al. (Morken et al. 2014), the authors extend model P12B by adding ADT-induced apoptosis of prostate cancer cells in addition to the inhibition of their growth and proliferation. Therefore, the model (hereafter M14) implements the per capita mortality of androgen-dependent and independent populations introduced in the previous section with the equation:

$${δ}_{i} \left({q}_{i}\right) = {δ}_{i m a x} \frac{{k}_{i / 2}^{2}}{{q}_{i}^{2} + {k}_{i / 2}^{2}} ,$$

where k_{i/2} for i ɛ {D, I} are the apoptosis and half-saturation levels for the dependent and independent populations, respectively. The SoE is considered in the form of:

$$\begin{matrix}\frac{d {n}_{D}}{d t} = \\ {n}_{D} \left(- {δ}_{D} - \frac{{δ}_{D m a x} {k}_{D δ / 2} {}^{2}}{{k}_{D δ / 2} {}^{2} + {q}_{{D}^{2}}} - \frac{{k}_{D I / 2} {}^{2} {μ}_{D I m a x}}{{k}_{D I / 2} {}^{2} + {q}_{{D}^{2}}} + {γ}_{max} \left(1 - \frac{{q}_{D m i n}}{{q}_{D}}\right)\right) + \\ \frac{{μ}_{I D m a x} {n}_{I} {q}_{i} {}^{2}}{{k}_{I D / 2} {}^{2} + {q}_{{I}^{2}}} , \\ \frac{d {n}_{I}}{d t} = \frac{{k}_{D I / 2} {}^{2} {μ}_{D I m a x} {n}_{D} {}^{2}}{{k}_{I D / 2} {}^{2} + {q}_{{D}^{2}}} + \\ {n}_{I} \left(- {δ}_{I} - \frac{{δ}_{I m a x} {k}_{I δ / 2} {}^{2}}{{k}_{I δ / 2} {}^{2} + {q}_{{I}^{2}}} - \frac{{μ}_{I D m a x} {q}_{i} {}^{2}}{{k}_{I D / 2} {}^{2} + {q}_{{I}^{2}}} + {γ}_{max} \left(1 - \frac{{q}_{I \text{min}}}{{q}_{I}}\right)\right) ,\end{matrix}$$

for qᵢ(t) ≠ 0 ∀t and i.c. n_{D}(t_{0D}) = n_{D0} and n_{I}(t_{0I}) = n_{I0}, together with the equivalent of above:

$$\begin{matrix}\frac{d {c}_{P S A}}{d t} = - {C}_{P S A} {δ}_{P S A} + {n}_{D} \left({γ}_{P S A 0} + \frac{{γ}_{P S A , D} {q}_{D} {}^{2}}{{k}_{P S A , D / 2} {}^{2} + {q}_{{D}^{2}}}\right) + \\ {n}_{I} \left({γ}_{P S A 0} + \frac{{γ}_{P S A , I} {q}_{I} {}^{2}}{{k}_{P S A , I / 2} {}^{2} + {q}_{{I}^{2}}}\right) ,\end{matrix}$$

with i.c. c_{PSA}(t_{0PSA}) = c_{PSA0}. Furthermore, the same notation as in the models by Portz et al. is followed and not repeated here.

### The Model in the Context of the Data

The analytical treatment is analogous to P12B but enriched in the dynamics variety for the extra parameters introduced in Eq. (32), although without changing equilibrium points. Due to the complexity of the model, analogous inference approximations to P12B have been used in this analysis. The model analysis did not report other notable features.

### Baez and Kuang 2016

The model by Baez and Kuang (Baez and Kuang 2016) presents a variant of the P12A model that is able to fit PSA and androgen dynamics, thus improving PSA trend forecasting. Two models are presented in the authors’ work and considered here. The first (hereafter B16A) is a single population model of cellular concentration n, and two equations are coupled with it, for δₘₐₓ the time-dependent (over a timescale ^{τ}δₘₐₓ) maximum baseline cell death rate and c_{PSA} the PSA concentration, that are modeled as:

$$\begin{matrix}\frac{d n}{d t} = n \left(- n δ - \frac{{k}_{n / 2} {δ}_{max}}{q + {k}_{n / 2}} + \frac{{γ}_{max} {q}_{min}}{q} + {γ}_{max}\right) , \\ \frac{d {δ}_{max}}{d t} = - {τ}_{{δ}_{max}} {δ}_{max} , \\ \frac{d {c}_{P S A}}{d t} = q \left({γ}_{P S A 1} n + {γ}_{P S A 0}\right) - {δ}_{P S A} {c}_{P S A} ,\end{matrix}$$

and a decoupled equation for androgen level:

$$\frac{d q}{d t} = γ \left({q}_{max} - q\right) - {γ}_{max} \left(q - {q}_{min}\right) ,$$

with n(t₀ₙ) = n₀, c_{PSA}(t_{0PSA}) = c_{PSA0}, δₘₐₓ(t_{0δmax}) = δ₀ₘₐₓ, and q(t_{0q}) = q₀ > 0 strictly. The quota q ≠ 0∀t is produced at a rate γ= γ₁Tₚₛ + γ₂

In the same work, the authors also presented a two-populations model tracking both sensitive n_{D} and independent n_{I}, cell evolution (hereafter B16B). By implementing their SoE within the approximation that all the cells have, on average, the same mass and density, the SoE can be recast in the form:

$$\begin{matrix}\frac{d {n}_{D}}{d t} = {n}_{D} \left(- \frac{{δ}_{D m a x} {k}_{D / 2}}{q + {k}_{D / 2}} - \frac{{k}_{D I / 2} {μ}_{D I max}}{q + {k}_{D I / 2}} + {γ}_{max} \left(1 - \frac{{q}_{D m i n}}{q}\right)\right) - {δ}_{D} {n}_{D} {}^{2} , \\ \frac{d {n}_{I}}{d t} = \frac{{k}_{D I / 2} {μ}_{D I max} {n}_{D}}{q + {k}_{D I / 2}} + {n}_{I} \left({γ}_{max} \left(1 - \frac{{q}_{I \text{min}}}{q}\right) - \frac{{δ}_{I m a x} {k}_{I / 2}}{q + {k}_{I / 2}}\right) - {δ}_{I} {n}_{I} {}^{2} , \\ \frac{d q}{d t} = - q \left({γ}_{2} + {γ}_{max} + {γ}_{1} {T}_{p s}\right) + \frac{{γ}_{max \left({q}_{D m i n} {n}_{D} + {1}_{I m i n} {n}_{I}\right)}}{{n}_{D} + {n}_{I}} + {q}_{max} \left({γ}_{2} + {γ}_{1} {T}_{p s}\right) , \\ \frac{d {c}_{P S A}}{d t} = q \left({γ}_{P S A 0} + {γ}_{P S A 1} \left({n}_{D} + {n}_{I}\right)\right) - {δ}_{P S A} {c}_{P S A} ,\end{matrix}$$

for nᵢ, i ∈ {D,I} never contemporaneously null, with initial conditions n_{D}(t_{0D}) = n_{D0}, n_{I}(t_{0I}) = n_{I0}, q(t_{0q}) = q₀, and c_{PSA}(t_{0PSA}) = c_{PSA0}. The maximum AD to AI mutation rate is given by µ_{DImax}. Furthermore, because AI cells, n_{I}, proliferate at lower androgen level it is assumed that q_{Imin} < q_{Dmin}, and δ_{Dmax} > δ_{Imax} because independent cells are less susceptible to apoptosis by androgen deprivation than sensitive cells.

### B16A in the Context of the Data

The decoupled quota equation presents an equilibrium at q_{eq} =

$$\frac{{γ}_{max} \left({q}_{min} - {q}_{max}\right)}{{γ}_{max} + \left({γ}_{2} + {γ}_{1} {T}_{p s}\right)} + {q}_{max} \text{when} {γ}_{max} + \left({γ}_{2} + {γ}_{1} {T}_{p s}\right) \neq 0 ,$$

belonging to the positive hyper-quadrant of the phase-space (i.e., it is of biological interest). The remaining set shows two equilibria at

$${\left\{n , {δ}_{max} , {c}_{P S A}\right\}}_{\text{eq}}^{\left(1\right)} = \left\{0 , 0 , \frac{{γ}_{\text{PSA,o}} {q}_{\text{eq}}}{{δ}_{\text{PSA}}}\right\} ,$$

which are always in the positive hyper-quadrant of interest and

$$\begin{matrix}{\left\{n , {δ}_{max} , {c}_{P S A}\right\}}_{\text{eq}}^{\left(2\right)} = \\ \left\{\frac{{γ}_{max} \left({q}_{\text{eq}} - {q}_{min}\right)}{{q}_{\text{eq}} δ} , 0 , \frac{δ {γ}_{\text{PSA,0}} {q}_{\text{eq}} + {γ}_{max} {γ}_{\text{PSA,1}} \left({q}_{\text{eq}} - {q}_{min}\right)}{{δ}_{\text{PSA}} δ}\right\} \\ \text{with} {q}_{\text{eq}} \neq 0 , {δ}_{\text{PSA}} \neq 0 \text{and} δ \neq \end{matrix}$$

0, which is also biologically meaningful. By studying the generalized eigenvalues, the first of the equilibrium presents three negative generalized eigenvalues, one of which is always positive (i.e., it is a saddle point); the second equilibrium point produces the eigenvalues

$${λ}_{1}^{\left(2\right)} = {γ}_{max} \left(\frac{{q}_{min}}{{q}_{\text{eq}}} - 1\right) , {λ}_{2}^{\left(2\right)} =$$

=

$$- {δ}_{\text{PSA}} , {λ}_{3}^{\left(2\right)} = - {τ}_{{δ}_{max}} \text{and} {λ}_{4}^{\left(2\right)} = - {γ}_{max} - \left({γ}_{2} + {γ}_{1} {T}_{p s}\right)$$

which are all always negative, thus representing a stable point of attraction.

Due to the stability of the second equilibrium (on- and off-treatment), it is worth investigating the proximity of the patients’ orbits to the equilibria on the Poincare sections involving the PSA concentration c_{PSA} obtained from Eq.(35). Nevertheless, the low quality of the likelihood, L(p) = Pr(D|p, I) , see above) in the Ω set of patients, demotivates further analysis. A single population n seems to not adequately capture disease progression, which remain the primary focus of the present disclosure, making the model less attractive for clinical implications and therefore not pushed forward here.

### B16B in the Context of the Data.

The model presents cubic dependence on q and quadratic on n_{D}. Throughout algebraic manipulators as Mathematica or MAPLE it is possible to show that the system characterizing equation for c_{PSA} is algebraic of order 12, whose complete numeric roots investigation is beyond the scope of the present disclosure. Only the null-equilibrium point of independent and dependent cells is selected for investigation. It is evident that n_{D} = 0 is an equilibrium for the first equation. Therefore, by assuming n_{D} = 0 (and n₁ > 0 strictly), the existence of two equilibria can be confirmed, the first located

$$\begin{matrix}\text{at} {\left\{{n}_{I} , q , {c}_{P S A}\right\}}_{\text{eq}}^{\left(1\right)} = \left\{0 , {q}_{max} + \frac{{γ}_{max} \left({q}_{I \text{min}} - {q}_{max}\right)}{{γ}_{max} + \left({γ}_{2} + {γ}_{1} {T}_{\text{ps}}\right)} , \frac{{γ}_{P S A 0}}{{δ}_{\text{PSA}}} {q}_{\text{eq}}\right\} , \\ \text{for} {γ}_{max} + \left({γ}_{2} + {γ}_{1} {T}_{\text{ps}}\right) \neq 0 \text{and} {δ}_{\text{PSA}} \neq 0 ,\end{matrix}$$

which is of biological interest. The second, algebraically more cumbersome, reduces its non-negativity condition to the simple one

$${δ}_{I m a x} + \frac{γ {γ}_{max} \left({q}_{I \text{min}} - {q}_{min}\right)}{{γ}_{max} {q}_{I \text{min}} + γ {q}_{max}} + \frac{γ {γ}_{max} \left({q}_{I \text{min}} - {q}_{min}\right)}{{k}_{I / 2} \left(γ + {γ}_{max}\right)} \leq 0 ,$$

that is verified over all studied patients.

Again, as explored in previous models, the existence of negative generalized eigenvalues of the Jacobian at the equilibria off-treatment, i.e., a point of equilibrium with an asymptotic constrained expansion of the tumoral cell population is explored. Despite the model complexity, it is easy to prove numerically that the Jacobian for both equilibrium points has at least one positive generalized eigenvalue, making these points saddle points that are not of interest to us.

### Elishmereni Et Al. 2016

The Elishmereni et al. (Elishmereni et al. 2016) model accounts for two dynamics: disease dynamics represented by PSA used as a proxy for tumor volume and the pharmacology dynamics combined with the emergence of resistant cells from androgen receptor-independent n_{I} and testosterone androgen receptor-dependent n_{IAR} mechanism. The PSA concentration c_{PSA} of interest to us, is governed by the following numerically highly complex SoE:

$$\begin{matrix}\frac{d {c}_{\text{PSA}}}{d t} = {\hat{c}}_{P S A} {γ}_{t P S A} min \left({γ}_{P S A} {c}_{P S A}^{K} , \frac{log 2}{{γ}_{P S A max}}\right) + \\ {η}_{T , P S A} {\left({c}_{\text{PSA}} - \frac{{\tilde{c}}_{\text{PSA}}}{2}\right)}^{+} \left({η}_{I , T} {R}_{I} {\hat{c}}_{P S A} + {n}_{T} - 1\right) , \\ \frac{d {n}_{T}}{d t} = \frac{{γ}_{T \left(1 - {\text{T}}_{\text{ps}}\right)}}{{η}_{H , T} H + 1} - {γ}_{T} {n}_{T ,} \\ \frac{d H}{d t} = T - \frac{{δ}_{T} {l}_{H}^{max} H {e}^{{R}_{T : A R}}}{{e}^{R} T : A R + {l}_{H}^{max}} , \\ \frac{d {R}_{T : A R}}{d t} = {γ}_{T : A R} T {\hat{R}}_{T : A R} , \\ \frac{d {R}_{I}}{d t} = {γ}_{I} T {\hat{R}}_{I} , \\ \frac{d \text{K}}{d t} = - {ρ}_{K} , \\ \frac{d \text{T}}{d t} = - {δ}_{T} T\end{matrix}$$

with c_{PSA}(t_{0PSA}) = c_{PSA0}, n_{T}(tₒₙ_{T}) = n_{T0}, H(t_{OH}) = H₀, R_{T:AR} (t_{0T:AR}) = R_{T:ARO}, R_{I}(t_{0I}) = R_{I0}, K(t_{0K}) = K₀, and T(t_{0T}) = T₀ with (x)⁺ = xθ(x) ramp/positive-function of the generic x, θ the previously introduced Heaviside step function. In the above equation γ_{PSAmax} is the limit to the PSA growth rate, _{ρK} the K growth rate, η_{T},_{PSA} the testosterone, T, effect on the PSA growth, γ_{T} the instantaneous rate of change in T, η_{H},_{T} the effect of intermediate components H, e.g., bound androgen receptor AR, on T, with same clearance rate δ_{T} . γ_{T:AR} is the increase resistance rate, γ_{I} the increase-resistance-rate for testosterone-AR independent paths R_{I,} and η_{I,T} rules the effect of R_{I} on the PSA growth. The growth rate of c_{PSA} is given by

$${γ}_{P S A} = \left\{\begin{matrix}1 & {c}_{P S A} > {c}_{t P S A} \\ {σ}_{P S A} + \left(1 - {σ}_{P S A}\right) \frac{{c}_{P S A}}{{c}_{t P S A}} & {c}_{P S A} \leq {c}_{t P S A} ,\end{matrix}\right)$$

where σ_{PSA} rules the steepness on the linear grown relation, c_{tPSA} the PSA threshold to switch in quiescent mode. Finally, control limits lᵢ i ∈ {PSA, H, n_{I}, n_{IAR}} are added by hand to handle system divergences with a “manual”-bounding scheme

$$\left({\hat{f}}_{i}\right) ≡ \frac{{\left({l}_{i max} - {f}_{i}\right)}^{+}}{{l}_{i max}}$$

for the generic function fᵢ).

In the practice the dynamics of the system is designed so that the instantaneous androgen rate of change γ_{T} is saturated by a control coefficient n_{T},_{PSA} through an intermediary delaying effect ruled by a delay modeling function H over the ADT therapy, T therapy-function with scale factor δ_{ADT} and a double mechanism for androgen independence cell population depending on n_{I,T}, and not depending on n_{I}, the androgen receptor (with the respective scale factor γ_{I} and γ_{T:AR}).

### The Model In The Context of the Data

The system has no equilibria influencing its dynamics, as evident from the 6th of Eq. 38. Further analysis is done to determine how well the model performs in the Bayesian model comparison.

### Zhang Et Al. 2017

Zhang et al. (Zhang et al., 2017) presents a three-population competition model, based on Lotka-Volterra (LV) dynamics, where androgen-dependent n_{D}, androgen producing nₚ, and androgen-independent cells n_{I}, are considered. Basing the approach on game theory, the authors derive a competition matrix α = aᵢⱼ i,j ∈ {D, I, P} based on the parametrization of growth rates γᵢ and carrying capacities kᵢ with i ∈ {D, I, P} resulting in this set of algebraic-differential equations:

$$\begin{matrix}\frac{d {n}_{D}}{d t} = {γ}_{D} {n}_{D} \left(1 - \frac{{α}_{11} {n}_{D} + {α}_{12} {n}_{p} + {α}_{13} {n}_{I}}{{n}_{p} \left(β - {T}_{p s} + 1\right)}\right) , \\ \frac{d {n}_{P}}{d t} = {γ}_{P} {n}_{p} \left(1 - \frac{{α}_{21} {n}_{D} + {α}_{22} {n}_{p} + {α}_{23} {n}_{I}}{{K}_{p}}\right) , \\ \frac{d {n}_{I}}{d t} = {γ}_{i} {n}_{I} \left(1 - \frac{{α}_{31} {n}_{D} + {α}_{32} {n}_{p} + {α}_{33} {n}_{I}}{{K}_{I}}\right) ,\end{matrix}$$

where ADT is modeled by the decreasing carrying capacity with β < 1 or supporting androgen-dependent cells with β > 1. The authors considered several constraints derived from the literature and researchers’ experience to shape the model parameter influence: αᵢᵢ = Ii, a₃₁ > α₂₁. α₃₂ > α₁₂. α₁₃ > α₂₃, α₁₃ > α₂₁, α₃₂ > α₃₁,and αᵢⱼ ∈ ]0,1[i ≠ j. Finally, the PSA dynamics is governed by:

$$\frac{d {c}_{P S A}}{d t} = {\sum }_{i ∈ \left\{D , P . I\right\}} {n}_{i} - δ {c}_{P S A} ,$$

with δ the PSA clearance rate.

### The Model in the Context of the Data

With the coupling of Eq. (41), the system presents four equilibria, but only two are of biological interest:

$${\left\{{n}_{D} , {n}_{P} , {n}_{I} , {c}_{P S A}\right\}}_{\text{eq}}^{\left(1\right)} = \left\{0 , {k}_{p} , 0 , \frac{{k}_{p}}{δ}\right\} ∈ {ℝ}_{0}^{4 +} \text{and} {\left\{{n}_{D} , {n}_{P} , {n}_{I} , {c}_{P S A}\right\}}_{\text{eq}}^{\left(2\right)} =$$

$$\left\{\begin{matrix}\frac{{k}_{P} \left(β - {α}_{12} - {T}_{p s} + 1\right)}{{α}_{21} \left(β - {α}_{12} - {T}_{p s} + 1\right) + 1} , \frac{{k}_{P}}{{α}_{21} \left(β - {α}_{12} - {T}_{p s} + 1\right) + 1} , \\ 0 \frac{{k}_{P} \left(α - {α}_{12} - {T}_{p s} + 2\right)}{δ + δ {α}_{21} \left(β - {α}_{12} - {T}_{p s} + 1\right)}\end{matrix}\right\}$$

where these ratios exist. For the first equilibrium, the eigenvalues of the Jacobian are positive in the n_{D}, nₚ and c_{PSA} phase-space and therefore of marginal interest. Vice versa, by setting α ≡ 1 + β, b ≡ β - a₁₂ + 1, d ≡ a₂₁(β - a₁₂ + 1) + 1 and e ≡ β + a₂₁(β - a₁₂ + 1)² - a₁₂ + 1 together with the discriminant squared Δ² = (eγ_{D} + βγ_{P} + γ_{P})² - 4adeγ_{D}γ_{P}, the four eigenvalues of the Jacobian can be written for the second equilibrium off-treatment as:

$${λ}_{1}^{\left(2\right) \text{off}} = - δ ,$$

$${λ}_{2}^{\left(2\right) \text{off}} = {γ}_{I} - \frac{{γ}_{I} {k}_{P} \left(b {α}_{31} + {α}_{32}\right)}{d {k}_{I}} ,$$

$${λ}_{3}^{\left(2\right) \text{off}} = - \frac{a {γ}_{P} + Δ + e {γ}_{D}}{2 a d}$$

and

$${λ}_{4}^{\left(2\right) \text{off}} =$$

$$\frac{Δ - a {γ}_{P} - e {γ}_{D}}{2 a d} ,$$

where the ratios exist, which are always negative for the fitted parameters, hence representing a stable equilibrium and opening the possibility to achieve an equilibrium off-treatment.

### Phan Et Al. 2019

The model (hereafter P19) presented by Phan et al. (Phan et al. 2019) is a variant of the work described herein (Baez and Kuang 2016) in which the third population of weakly dependent cells, n_{wD}, is added to investigate the influence of extra degrees of freedom added by the new population. The death term is also adapted from Eq. (33). Retaining the notation used herein, the model can be recast in the following form:

$$\begin{matrix}\frac{d {n}_{D}}{d t} = \\ {n}_{D} \left(- \frac{{δ}_{D max} {k}_{D / 2}}{q + {k}_{D / 2}} - \frac{2 {k}_{D I / 2} {μ}_{D \text{Im} a x}}{q + {k}_{D I / 2}} + {γ}_{max} \left(1 - \frac{{q}_{D min}}{q}\right)\right) + \\ \frac{{k}_{D I / 2} {μ}_{D \text{Im} a x} {n}_{w D}}{q + {k}_{D I / 2}} - {δ}_{D} {n}_{D}^{2} \\ \frac{d {n}_{w D}}{d t} = \\ {n}_{w D} \left(- \frac{2 {k}_{D I / 2} {μ}_{D \text{Im} a x}}{q + {k}_{D I / 2}} - \frac{{δ}_{w D max} {k}_{w D / 2}}{q + {k}_{w D / 2}} + {γ}_{max} \left(1 - \frac{{q}_{w D min}}{q}\right)\right) + \\ \frac{{k}_{D I / 2} {μ}_{D \text{Im} a x} {n}_{D}}{q + {k}_{D I / 2}} - {δ}_{w D} {n}_{w D}^{2} \\ \frac{d {n}_{I}}{d t} = \\ \frac{{k}_{D I / 2} {μ}_{D \text{Im} a x} \left({n}_{D} + {n}_{w D}\right)}{q + {k}_{D I / 2}} + {n}_{I} \left({γ}_{max} \left(1 - \frac{{q}_{\text{Im} i n}}{q}\right) - \frac{{δ}_{\text{Im} a x} {k}_{I / 2}}{q + {k}_{I / 2}}\right) - {δ}_{I} {n}_{I}^{2} \\ \frac{d q}{d t} = \\ q \left(- \left({γ}_{2} + {γ}_{1} {T}_{p s}\right) - {γ}_{max}\right) + \frac{{γ}_{max} \left({q}_{D min} {n}_{D} + {q}_{\text{Im} i n} {n}_{I} + {q}_{w D min} {n}_{w D}\right)}{{n}_{D} + {n}_{I} + {n}_{w D}} + \\ {q}_{max} \left({γ}_{2} + {γ}_{1} {T}_{p s}\right) \\ \frac{d {c}_{P S A}}{d t} = q \left({γ}_{P S A 0} + {γ}_{P S A 1} \left({n}_{D} + {n}_{I} + {n}_{w D}\right)\right) - {δ}_{P S A} {c}_{P S A}\end{matrix}$$

with initial conditions n_{D}(t_{0D}) = n_{D0}, n_{wD} (t_{0wD}) = n_{wD0}, n_{I}(t_{0I},) = n_{I0}, q(t_{0q}) = q₀, c_{PSA}(t_{0PSA}) = c_{PSA0} together with the required biological inequalities q_{Dmin} > q_{wDmin} and q_{Dmin} > q_{Imin}.

### P19 in the Context of The Data.

The idea of a third population is not new and already advanced with success in the model by Hirata et al. 2010. Nevertheless, the structure of the equations is very different from the Hirata et al. model above, with significantly more parameters not readily justifiable within the present dataset quality. Similar considerations were already worked out by Phan et al. Only that the complexity of the analysis, already evident as detailed herein, is pushed further in this context, where only numerical investigation is available for equilibria and stability. The only off-treatment equilibrium accessible by the orbits is the one for

$${\left\{{n}_{D} , {n}_{w D} , {n}_{I} , q , {c}_{P S A}\right\}}_{\text{eq}}^{\text{off}} = \left\{0 , 0 , 0 , \frac{{γ}_{max} {q}_{\text{Imin}} + {γ}_{2} {q}_{max}}{{γ}_{2} + {γ}_{max}} ,\right)$$

$$\left(\frac{{γ}_{\text{PSA} 0} \left({γ}_{max} {q}_{\text{Imin}} + {γ}_{2} {q}_{max}\right)}{{δ}_{\text{PSA}} \left({γ}_{2} + {γ}_{max}\right)}\right\} \text{and} {δ}_{P S A} \neq 0$$

which is always positive with always negative eigenvalues

$${λ}_{1}^{\text{off}} =$$

$$- {γ}_{2} - {γ}_{max} \text{and} {γ}_{2}^{\text{off}} = - {δ}_{P S A ⋅}$$

This is of limited biological interest as it is not compatible with the irreversibility nature of n_{I}, if not by surgical castration.

### Brady-Nicholls Et Al. 2020

The Brady-Nicholls et al. (Brady-Nicholls et al. 2020) model (hereafter B20) is based on the hypothesis that prostate cancer stem cells’ enrichment induces resistance. The model correlates stem cell proliferation with serum PSA through SoE for the prostate cancer stem cells nₛ, the non-stem (differentiated) cells n_{D}, and for PSA serum concentration c_{PSA}. The system is reported in the following way:

$$\begin{matrix}\frac{d {n}_{S}}{d t} = \frac{{p}_{S} log \left(2\right) {n}_{S} {}^{2}}{{n}_{D} + {n}_{S}} , \\ \frac{d {n}_{D}}{d t} = log \left(2\right) {n}_{S} \left(1 - \frac{{p}_{S} {n}_{S}}{{n}_{D} + {n}_{S}}\right) - {δ}_{D} {T}_{p s} {n}_{D} , \\ \frac{d {c}_{P S A}}{d t} = {γ}_{\text{PSA}} {n}_{D} - {δ}_{\text{PSA}} {c}_{\text{PSA}} ,\end{matrix}$$

with initial conditions nₛ(t₀ₛ) = n_{S0}, n_{D}(t_{0D}) = n_{D0} and c_{PSA}(t_{0PSA}) = c_{PSA0}. It is assumed that stem cells divide at rate log(2), and the division is either symmetric yielding two stem cells (Enderling 2015) or asymmetric, where the stem cell produces one stem and one differentiated cell. The parameter that governs this effect is pₛ. The PSA differentiated cell production rate and PSA clearance rate are given by γ_{PSA} and δ_{PSA}, respectively and Tₚₛ is the patient-specific treatment function.

### The Model In The Context of The Data

The SoE presents an infinite set of equilibrium points when off-treatment Tₚₛ(t) = 0 in the intersection of the plane nₛ(t) = 0 with the plane given by

$${c}_{P S A} \left(t\right) = \frac{{γ}_{\text{PSA}} {n}_{D} \left(t\right)}{{δ}_{\text{PSA}}}$$

conditional to n_{D} ≠ 0 and δ_{PSA} ≠ 0 and the generalized eigenvalues of the Jacobian results in a double zero generalized eigenvalue λ₁ = 0, λ₂ = 0 and a third negative eigenvalue λ₃ = — δ_{PSA}. Standard center manifold computation (Wiggins 2003) shows slow-2D-manifold dynamics that can be integrated to prove that the equilibria are unstable, and therefore not of interest.

### Bayesian Model Comparison

Maybe the most vital point of the Bayesian framework, and the reason for its increasing popularity, is its innate model comparison ability, based on logic as an instrument for selection. This feature is exploited here using the Bayesian factor to compare the different models in their ability to simulate the data. It should be noted that this framework innate penalizes models based on the number of parameters required. This phenomenon is sometimes referred to as the Occam’s razor factor (Jefferys and Berger 1992).

Starting from the classical Bayesian theorem, the Bayes factor βᵢⱼ for PSA model Mᵢ over the PSA model Mⱼ is computed as a ratio of the probabilities of the two models (the odd-ratio, Oᵢⱼ)

$${O}_{i j} = \frac{\text{Pr} \left({M}_{i} , I\right) \text{Pr} \left(D \left|{M}_{i} , I\right)\right)}{\text{Pr} \left({M}_{j} , I\right) \text{Pr} \left(D \left|{M}_{j} , I\right)\right)} = \frac{\text{Pr} \left({M}_{i} , I\right)}{\text{Pr} \left({M}_{j} , I\right)} {β}_{i j} ,$$

such that, because

$${\sum }_{i = 1}^{{N}_{m}} \text{Pr} \left({M}_{i} \left|D , I\right)\right) = 1$$

(with Nₘ number of models to compare) if interested in how a model, say M_{1,} compares to the other models Mⱼ, the following can be arrived at

$$\text{Pr} \left({M}_{1} \left|D , I\right)\right) = \frac{{O}_{i 1}}{{\sum }_{j = 1}^{{N}_{m}} {O}_{j 1}} .$$

The equation is implemented to compare one patient at a time in one model against all the other models individually. For example, in implementing the comparison between M₁, and every other M₂ as

$$\text{Pr} \left({M}_{2} \left|D , I\right)\right) = \frac{1}{1 + {O}_{21}^{- 1}} ,$$

and proceed iteratively.

The Laplace approximation framework is explored under the assumption of equally-prioritized models, i.e., assuming that no previous preference can be accorded to any of the PSA models considered. The asymptotic approximation can be exploited (Murphy 2012; Theodoridis 2015) to the global-likelihood, i.e., the evidence of the i^{th} model, Pr(D|Mᵢ), writing

$$\begin{matrix}\text{Pr} \left(D \left|{M}_{i}\right)\right) = \int d \text{pPr} \left(\text{p} \left|M , I\right)\right) L \left(\text{p} \left|I\right)\right) \\ ≅ \text{Pr} \left(\hat{\text{p}} \left|{M}_{i}\right)\right) L \left(\hat{\text{p}}\right) \sqrt{det \left(\text{F} \left(\text{p}\right)\right)} ,\end{matrix}$$

with F being the information matrix introduced herein. A classical result of Bayesian analysis is to consider the limit of the previous expression, but for an increased number of data points (Nₚ → ∞) and flat priors, i.e., to compute the popular BIC index against AIC (Akaike 1974; Schwarz 1978). As the number of patient data points is often limited Nₚ « ∞ and make explicit use of priors, BIC or AIC indices are not justifiable for model comparison. Instead, a model-of-model function (Pasetto et al., 2021) is built to encode prior information as soon as available. Furthermore, as introduced above, the Laplace approximation is verified with fully numerical integration based on nested sampling algorithms (Skilling 2004; Mukherjee et al. 2006; Feroz and Hobson 2008), i.e., a numerical technique designed explicitly to compute the global-likelihood of models with different degree of freedom.

### Single Patient Comparison Results

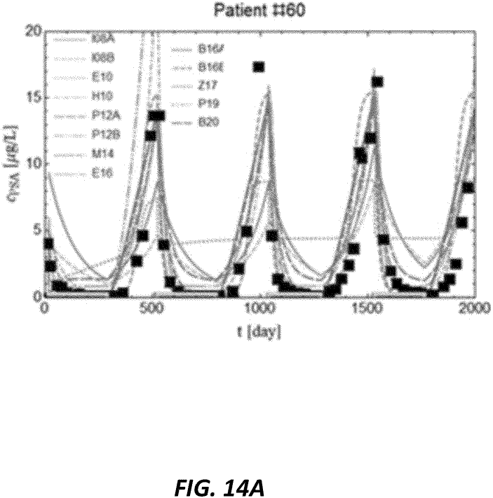

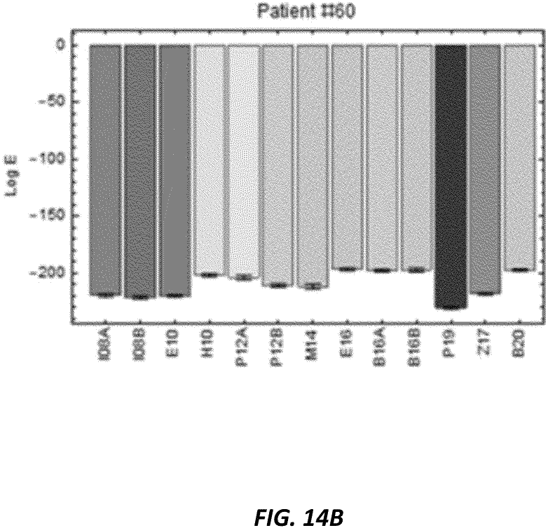

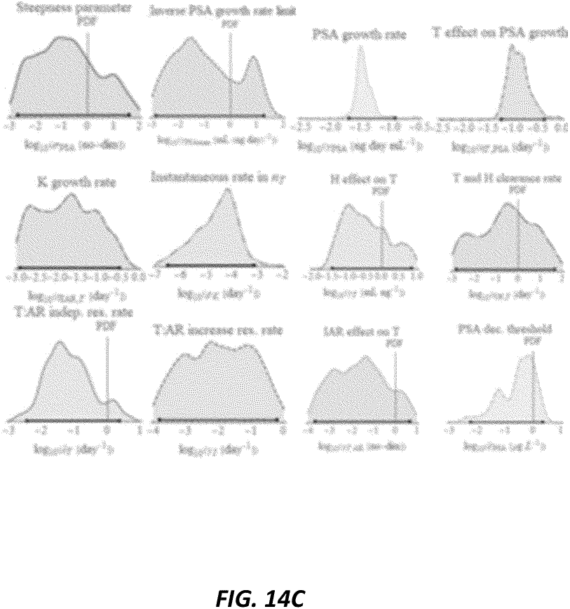

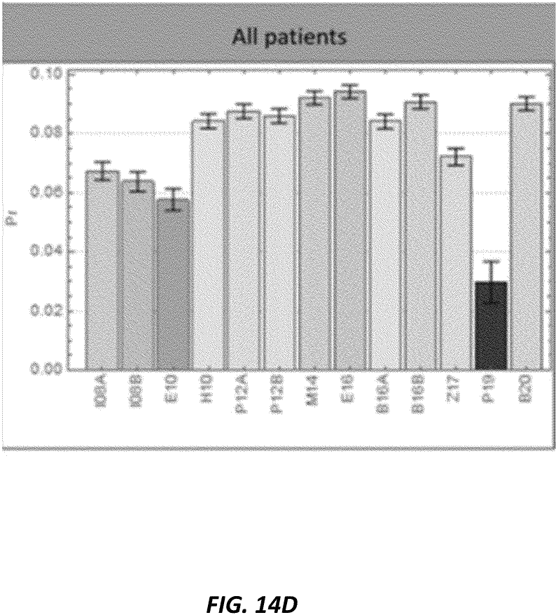

FIG. 14A shows an example of the quality of the model calibration achieved by Bayesian posterior inference introduced herein applied to the parameter inference problem to all the models. The simulated disease dynamics vary significantly between the different models, and discrepancies between different models and patient data may indicate likely or unlikely biological mechanisms driving individual patients’ resistance.

Model evidence (FIG. 14B) demonstrates that no single model represents all patient data accurately, suggesting that several different biology drive individual patients’ responses or that no model correctly faces the PSA problem. It may also imply that the PSA dynamics alone may be insufficient to discriminate between the different biological models. For some patients, model selection identifies models with a higher probability than others, but selection varies on a per-patient basis. As a classical proof-of-concept of the Bayesian technology employed, for the best performing model, E16, for patient #60 the unnormalized posterior marginalized PDF for each parameter in FIG. 14C is reported. The PDFs are almost unimodal (but not for all parameters), suggesting that this model represents the patient best and that the Laplace approximation could be justified. The credible intervals for the log parameters are also plotted and superimposed to the x-axis.

### Overall Model Selection

The Bayesian log-likelihood performance is calculated for all the patients for each model (FIG. 14D), resulting in the Elishmereni et al. 2016 model marginally performing better on most patients. This result does not surprise us, as it is a model designed on clinical necessities, i.e., it was crafted with careful handling of the treatment. Nevertheless, as mentioned before, in the case of model comparison on a patient-to-patient basis, a model that performed statistically better than the others could not be identified, thereby indicating the correct biological mechanics governing PSA dynamics. FIG. 14D shows that E16 is preferred only on 10% of the patients, and eight of the 13 models have scores above the 8%.

This work considers several mathematical models to simulate PSA dynamics of prostate cancer response to IADT in a prospective clinical trial. Bayesian continuous and discrete inference abilities are exploited to interpret the data and identify the model with the highest likelihood of simulating the clinically observed dynamics. Using the PSA biomarker and the comparison between the different models, 1) several models can be identified that can separate responding patients and patients that develop resistance to intermittent ADT through the model-fitting, 2) Bayesian model comparison demonstrated that the model by Elishmereni et al. 2016 performed slightly better than the others, i.e., as a better representative of most patients in the trial. Nevertheless, as evidence in the example of FIG. 14C, the marginalized posterior PDF is often not all optimally single-peaked, casting shadows in an attempt to use this model to solve forecast problems. The models’ inference has been used to evaluate the possible connection with their underpinned biology, the potentiality and limitation of the models’ forecasting ability to predict clinical PSA trends in a follow-up paper is explored (Pasetto et al. 2021, in preparation).

The models analyzed herein synonymously use longitudinal PSA data to infer biological mechanisms underlying the observed PSA dynamics. PSA alone limited the potentiality of the presented approach and did not identify a single dominant model. Further information is necessary to simulate accurately and ultimately predict patient-specific PSA trajectories and the corresponding biological drivers of resistance. PSA alone might not be a helpful biomarker due to several dominant environmental factors outside the models’ scopes that influence its evolution under treatment. The use of PSA as a surrogate marker for prostate cancer burden is indeed controversial. Overexpression of the PCA3 gene obtained from the mRNA in urine samples is proposed to be more suited to monitoring the cancer evolution (Bussemakers et al. 1999, p. 3; Laxman et al. 2008; Neves et al. 2008; Hessels and Schalken 2009, p. 3; Borros 2009).

Two alternative directions might be taken to improve understanding of the PSA as a prostate cancer monitor biomarker. From one side, a deeper understanding of the connection between PSA and tumor burden throughout model investigation might present the opportunity for a new class of models. Recently the role of immature blood vessels formed under angiogenesis cues has been investigated to decrypt the relation between an increased tumor burden contemporaneous to decreasing PSA concentration (Barnaby et al. 2021). Additionally, models that include both PSA and androgen concentrations might present some advantages in the future. The modest but significant evidence of the E16 model over the other models might indicate a more important relevance of the dormancy whose biology and mathematics are worth undoubtedly deeper understanding.

Exploring PSA model probability distributions to disentangle responsive and resistant patient cohorts in a clinical setting could be investigated through its cross-correlations with PCA3 biomarkers. Such cross-correlation would provide independent verification of the analytical findings herein that remain, for the moment, data-driven and, therefore, entirely dependent on the one dataset that was utilized in all discussed models.

Alternatively, PSA could be a perfect biomarker, but inter-patient heterogeneity in resistance mechanisms may disallow identifying a single model for all patients. Additionally, different resistance mechanisms may evolve in an individual patient, with their respective contribution to the observed response dynamics changing during therapy. More complex models and dynamic adaptive weighting of different variables, terms, and parameters may be necessary. Such models, however, would be non-identifiable with the presently available data. A close dialogue between biologists, statisticians, and mathematical and genitourinary oncologists may help identify which data should be collected in future clinical studies to help detangle the complex prostate cancer response dynamics to intermittent ADT.

While the Bayesian framework is an invaluable tool to estimate model parameters and fit model dynamics to clinical measurements, the goodness of a fit informs neither the reliability of the estimated parameters nor the likelihood of a model representing the data chosen for the valid biological reason. Relatively invariant PSA profiles can be obtained for a significant range in each parameter, as it is the case of a weakly sensitive - highly non-identifiable parameter. This fact is often omitted in the modeling literature, where results are often presented without structural or practical identifiability analysis. Many of the herein discussed models have not demonstrated structural identifiability, hence jeopardizing the attempt to claim the inference’s practical identifiability herein. Nevertheless, a model’s value may also be found in its interpretative role (Enderling and Wolkenhauer 2021). The complexity of the mechanism involved in the biological responses to intermittent ADT can be captured correctly for a single patient but fail for others. Therefore, the model comparison is not intended to provide an absolute ranking; instead, it provides an instrument to explore the different biological mechanisms implemented in mathematical models in clinically observed treatment response and progression dynamics.

### Supplement

A sensitivity analysis for all the models included here was performed. However, as this analysis overlaps with the original papers’ work, those results are not included here. The sensitivity is justified for several reasons: 1. to understand the dependence of results on the parameters. For example, if the possibility to split between relapsing (Ω) and not to relapse (_{¬}Ω) patients by exploiting some specific model parameter combination can be claimed, then the robustness of our result worth be investigated on the same parameter sensitivity to assign it the correct relevance and to evaluate its possibility to be applied to clinical tumor forecasting. 2. The technique implemented for the sensitivity analysis investigates the parameter’s sensitivity and the best-fit orbital integration, i.e., over the available longitudinal data. This approach enhances our understanding of when a particular Ω / _{¬}Ω segregating technique is more useful during or off treatment, with consequent indications on the role that a model splitting potentiality might or might not have (and when) on a per-patient base. 3. Continuous but not differentiable functions might need particular attention in the computation of the sensitivities because of their definition as the Jacobian matrix’s function. This approach represents a current research field often omitted in the mathematical oncology literature and worth being brought to light.

Therefore, in what follows, the Direct Differential Method (DDM) for sensitivity analysis (Gu and Wang 2013) is exploited to track the time dependence of the sensitivity

$${S}_{i j} ≡ \frac{\partial {x}_{i} \left(t , \hat{p}\right)}{\partial {p}_{j}} ,$$

where in general it is xᵢ = c_{PSA} and pⱼ the generic parameter dependent on the particular model in the exam. For a generic vector field

$$\frac{\partial \text{x} \left(\text{p} \text{;} t\right)}{\partial t} = \text{f} \left(\text{x,} \text{p}\right) \text{with} \text{x} \left({t}_{0} , \text{p}\right) = {\text{x}}_{0}$$

the integration of the SoE defining the model is coupled with:

$$\frac{\partial {S}_{i j} \left(t , \hat{p}\right)}{\partial t} = \frac{\partial {f}_{i} \left({x}_{k} \left(t , \hat{p}\right) , \hat{p}\right)}{\partial {x}_{k} \left(t , \hat{p}\right)} {S}_{k j} \left(t , \hat{p}\right) + \frac{\partial {f}_{i}}{\partial {p}_{j}} .$$

Generalized sensitivity (Stechlinski et al. 2018), based on the concept of generalized derivative for non-smooth c_{PSA} profiles (Clarke 1990) and used because of the loss of differentiability at the bifurcation points Tₚₛ = {0,1} on the treatment parameter, has also been considered. The DDM analysis is not reported if not relevant to strengthen specific results and the reader is referred to the original model paper for general sensitivity analysis of the presented models.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method for tumor forecasting, comprising:
inputting a plurality of patient data for a patient into a multi-model framework;
predicting, using the multi-model framework, a probability of a given treatment producing a given outcome for the patient; and
outputting an assessment for the given treatment.

2. The method of claim 1, wherein the multi-model framework comprises a Bayesian statistical model.

3. The method of claim 2, wherein the Bayesian statistical model is configured to analyze respective predictions of a plurality of models of the multi-model framework.

4. The method of claim 1, wherein the patient data comprises at least one of demographic data, clinical data, laboratory data, histological feature data, comorbidity data, and medication data.

5. The method of claim 1, wherein the given treatment comprises surgery, radiotherapy, chemotherapy, immunotherapy, or combinations thereof.

6. The method of claim 1, wherein the given outcome comprises at least one of tumor burden, tumor local control, progression-free survival for a period of time, and relapse-free survival for a period of time.

7. The method of claim 1, wherein the multi-model framework is implemented as a cloud-computing service or system.

8. The method of claim 1, further comprising recommending the given treatment for the patient.

9. The method of claim 8, further comprising administering the given treatment to the patient.

10. An apparatus comprising at least one processor, at least one memory including computer program code for at least one program, and a network interface, the at least one memory and the computer program code configured to, with the at least one processor, cause the apparatus to:
input a plurality of patient data for a patient into a multi-model framework;
predict, using the multi-model framework, a probability of a given treatment producing a given outcome for the patient; and
output an assessment for the given treatment.

11. The apparatus of claim 10, wherein the multi-model framework comprises a Bayesian statistical model.

12. The apparatus of claim 11, wherein the Bayesian statistical model is configured to analyze respective predictions of a plurality of models of the multi-model framework.

13. The apparatus of claim 10, wherein the patient data comprises at least one of demographic data, clinical data, laboratory data, histological feature data, comorbidity data, and medication data.

14. The apparatus of claim 10, wherein the given treatment comprises surgery, radiotherapy, chemotherapy, immunotherapy, or combinations thereof.

15. The apparatus of claim 10, wherein the given outcome comprises at least one of tumor burden, tumor local control, progression-free survival for a period of time, and relapse-free survival for a period of time.

16. The apparatus of claim 10, wherein the multi-model framework is implemented as a cloud-computing service or system.

17. A computer program product comprising at least one non-transitory computer-readable storage medium having computer-executable program code portions stored therein, the computer-executable program code portions comprising program code instructions, the computer program code instructions, when executed by a processor of a computing entity, are configured to cause the computing entity to at least:
input a plurality of patient data for a patient into a multi-model framework;
predict, using the multi-model framework, a probability of a given treatment producing a given outcome for the patient; and
output an assessment for the given treatment.

18. The computer program product of claim 17, wherein the multi-model framework comprises a Bayesian statistical model.

19. The computer program product of claim 18, wherein the Bayesian statistical model is configured to analyze respective predictions of a plurality of models of the multi-model framework.

20. The computer program product of any one of claim 17, wherein the given treatment comprises surgery, radiotherapy, chemotherapy, immunotherapy, or combinations thereof, and wherein the given outcome comprises at least one of tumor burden, tumor local control, progression-free survival for a period of time, and relapse-free survival for a period of time.
